(19) United States
(12) Patent Application Publication
(10) Pub. No.: US 2023/0228744 A1
(43) Pub. Date: Jul. 20, 2023

(54) DRY EYE DISEASE BIOMARKERS AND THEIR USE FOR TREATMENT

(71) Applicant: Aldeyra Therapeutics, Inc., Lexington, MA (US)

(72) Inventors: Todd BRADY, Carlisle, MA (US); Stephen Gitu MACHATHA, Wilmington, MA (US); Adam BROCKMAN, Arlington, MA (US)

(21) Appl. No.: 18/007,661

(22) PCT Filed: Jun. 4, 2021
(86) PCT No.: PCT/US2021/035948

§ 371 Date: Dec. 1, 2022

Related U.S. Application Data
(60) Provisional application No. 63/034,935, filed on Jun. 4, 2020.

(51) Int. Cl. G01N 33/53; G01N 33/68; A61K 31/47; A61K 9/00; A61P 27/04; A61K 47/40

(52) U.S. Cl. CPC G01N 33/5308; G01N 33/6893; A61K 31/47; A61K 9/0048; A61P 27/04; A61K 47/40

(57) ABSTRACT

The present invention relates to use of biomarkers of dry eye disease, and use of the biomarkers for selection of subjects for treatment and treatment of dry eye disease.

### CROSS-REFERENCE TO RELATED APPLICATIONS

This application claims the benefit of U.S. Provisional Patent Application No. 63/034,935, filed Jun. 4, 2020; the entirety of which is hereby incorporated by reference.

### TECHNICAL FIELD

The present invention relates to biomarkers for dry eye disease and use of the biomarkers for treatment of the disorder.

### BACKGROUND

Dry eye disease is a complex disease that results in ocular discomfort, visual disturbance, and tear film instability, which create the potential for damage to the ocular surface. It is characterized by increased osmolarity of the tear film and inflammation of the ocular surface. Estimates of the prevalence of dry eye disease vary considerably, depending on the criteria used to define the disease, but in the United States (U.S.), it has been estimated that as many as 20 million adults in the U.S. have dry eye disease. It has been projected that there will be a 40% increase in number of patients affected by 2030 (Schaumberg, Advances in Experimental Medicine and Biology, 2002, 506:989-98; Schaumberg, American Journal of Ophthalmology, 2003, 136:318-26; Schaumberg, Archives of Ophthalmology, 2009, 127:763-8). With the aging population in the U.S. and other countries of the developed world, and increasing computer use, dry eye disease is expected to become more prevalent. Thus, finding effective treatments is becoming more important (Brewitt, Survey of Ophthalmology, 2001, 45 Suppl 2:S199-202).

Aldehydes are reactive organic molecules that bind to proteins, carbohydrates, lipids and nucleic acids (Esterbauer, Free Radical Biology and Medicine, 1991, 11(1):81-128). Free aldehydes—aldehydes not sequestered or otherwise protected in specific metabolic processes—can be toxic, and aldehyde binding to cellular constituents can lead to inflammation (Yadav, Oxidative Medicine and Cellular Longevity, 2013, Volume 2013, Article ID 690545), molecular dysfunction (O'Brien, Critical Reviews in Toxicology, 2005, 35(7):609-62), and the accumulation of indigestible metabolites, such as lipofuscin components in the retina (Boyer, J Biol Chem., 2012, 287:22276-86).

In biological systems, aldehydes are formed by a variety of processes, including the oxidation of alcohols, polyamine and glucose metabolism, and oxidative stress. In some disease states, aldehyde concentrations may be increased. Increases in aldehyde concentrations has been described in a variety of inflammatory ocular diseases, including pterygium, Behcet's Disease, Sjögren's Syndrome, anterior uveitis, and dry eye disease (Sandikci, Acta Dermato-Venereologica, 2003, 83(5): 342-6; Cejkova, Histology and Histopathology, 2007, 22(9):997-1003; Balci, Molecular Vision, 2011, 17: 443-7; Turk, Ocular Immunology and Inflammation, 2014, 22(2):127-32; Choi, Current Eye Research, 2016, 41(9): 1143-9).

### SUMMARY

In some aspects, the present disclosure relates to use of biomarkers for dry eye disease (DED), also referred to herein as dry eye syndrome (DES; the two terms are used interchangeably), for use in assessing efficacy of treatments for dry eye disease and in the treatment of dry eye disease. In some embodiments, the biomarkers are also useful in the selection of patients for treatment of dry eye disease.

In one aspect, the present disclosure provides a method of assessing the effectiveness of an aldehyde trapping agent in treating dry eye disease in a patient. In some embodiments, the method comprises administering an aldehyde trapping agent to a patient with dry eye disease; measuring the level of an aldehyde marker of oxidative stress present in the eye of the patient; and comparing the measured level of the aldehyde marker of oxidative stress to level of aldehyde marker of oxidative stress in an appropriate control, wherein a reduction in level of aldehyde marker of oxidative stress indicates effectiveness of the aldehyde trapping agent in treating dry eye disease.

In another aspect, the present disclosure provides a method of assessing effectiveness of an aldehyde trapping agent in treating ocular inflammation in a subject, comprising: administering an aldehyde trapping agent to a subject with ocular inflammation; measuring the level of an aldehyde marker of oxidative stress present in the eye of the subject; and comparing the measured level of the aldehyde marker of oxidative stress to level of aldehyde marker of oxidative stress in an appropriate control; wherein a reduction in level of aldehyde marker of oxidative stress indicates effectiveness of the aldehyde trapping agent in treating ocular inflammation.

In another aspect, the present disclosure provides a method of treating ocular inflammation in a subject comprising:
(i) measuring the level of an aldehyde marker of oxidative stress in the eye of a subject with ocular inflammation prior to treatment;
(ii) treating the subject with an aldehyde trapping agent, wherein the aldehyde trapping agent is reproxalap and wherein the reproxalap is administered topically to the eye; and
(iii) measuring the level of the aldehyde marker of oxidative stress in the eye of the subject following treatment;

wherein the subject is treated with a lower dosing frequency of reproxalap for a reduction of greater than about 20% in the measured level of the aldehyde marker of oxidative stress, and wherein the subject is treated with the same or higher dosing frequency of reproxalap for a reduction of about 20% or less in the measured level of the aldehyde marker of oxidative stress.

In some embodiments, the ocular inflammation is associated with dry eye disease, allergic conjunctivitis, pterygium, Behcet's Disease, Sjögren's Syndrome, or uveitis (including, for example, anterior uveitis). In some embodiments, the ocular inflammation is associated with a corneal disease (e.g., dry eye syndrome, cataracts, keratoconus, bullous and other keratopathy, and Fuch's endothelial dystrophy), other ocular disorders or conditions (e.g., allergic conjunctivitis, ocular cicatricial pemphigoid, conditions associated with PRK healing and other corneal healing, and conditions associated with tear lipid degradation or lacrimal gland dysfunction), and other ocular conditions associated with high aldehyde levels as a result of inflammation (e.g., uveitis, scleritis, ocular Stevens Johnson Syndrome, ocular rosacea (with or without meibomian gland dysfunction)). In some embodiments, the ocular inflammation is associated with macular degeneration, such as age-related macular degeneration (“AMD”), or Stargardt's disease.

In another aspect, the present disclosure provides a method of using an aldehyde marker of oxidative stress for treating a patient with dry eye disease. In some embodiments, a method of treating dry eye disease in a patient comprises: measuring the level of an aldehyde marker of oxidative stress in the eye of a patient with dry eye disease prior to treatment; treating the patient with an aldehyde trapping agent, wherein the aldehyde trapping agent is reproxalap and wherein the reproxalap is administered topically to the eye; and measuring the level of the aldehyde marker of oxidative stress in the eye of the patient following treatment; wherein the patient is treated with a lower dosing frequency of reproxalap for a reduction of greater than about 20% in the measured level of the aldehyde marker of oxidative stress, and wherein the patient is treated with the same or higher dosing frequency of reproxalap for a reduction of about 20% or less in the measured level of the aldehyde marker of oxidative stress.

In another aspect, the present disclosure provides a method of selecting a subject for treatment of ocular inflammation, comprising: measuring the level of an aldehyde marker of oxidative stress in an eye of a subject suspected of having ocular inflammation, wherein a measured level of at least about 2 fold or greater level of the aldehyde marker of oxidative stress as compared to level of aldehyde marker of oxidative stress in subjects without ocular inflammation is indicated for treatment.

In another aspect, the present disclosure provides a method of identifying or selecting a patient with dry eye disease for treatment. In some embodiments, a method of selecting a patient for treatment of dry eye disease comprises: measuring the level of an aldehyde marker of oxidative stress in an eye (for example, in the tears) of a patient suspected of having dry eye disease, wherein a measured level of at least about 2-fold higher level of the aldehyde marker of oxidative stress as compared to the level of aldehyde marker of oxidative stress in patients without dry eye disease is indicated for treatment.

In some embodiments, the method further comprises the step of treating the dry eye disease if the level of the aldehyde marker of oxidative stress is indicated for treatment, wherein the treatment comprises administering to the patient an effective amount of reproxalap.

In some embodiments, the method further comprises the step of treating the dry eye disease by administering to the patient an effective amount of reproxalap if the patient exhibits in at least one of the patient's eyes the measured level of at least about 2.5-fold higher of the aldehyde marker of oxidative stress as compared to level of aldehyde marker of oxidative stress in patients without dry eye disease.

In some embodiments, the level of the aldehyde marker of oxidative stress is indicated for treatment if it is about 2-fold to 6-fold higher than the level of aldehyde marker of oxidative stress in patients without dry eye disease. In some embodiments, the level of the aldehyde marker of oxidative stress is indicated for treatment if it is about 2-fold to 5-fold, about 2.5-fold to 4.5-fold, about 3-fold to 4-fold, or about 3.5-fold to 4-fold higher than the level of aldehyde marker of oxidative stress in patients without dry eye disease.

In some embodiments, the level of the aldehyde marker of oxidative stress is indicated for treatment if it is about 2-fold, about 2.5-fold, about 3-fold, about 3.5-fold, about 4-fold, about 4.5-fold, about 5-fold, about 5.5-fold, or about 6-fold higher than the level of aldehyde marker of oxidative stress in patients without dry eye disease.

In some embodiments, the method of selecting or identifying a patient for treatment of dry eye disease is for treatment with an aldehyde trapping agent. In some embodiments, the aldehyde trapping agent is reproxalap.

In some embodiments, the aldehyde marker of oxidative stress in the methods herein is malondialdehyde or 4-hydroxynonenal. In some embodiments, the level of the aldehyde marker of oxidative stress measured is in the form of adducts of the aldehyde marker of oxidative stress present in the eye. In some embodiments, the adducts comprise stable adducts formed with proteins in the eye. In some embodiments, the adducts comprise adducts formed with malondialdehyde. In some embodiments, the adducts comprise adducts formed with 4-hydroxynonenal. In some embodiments, a sample of tear obtained from a patient is used to measure the level of the adducts.

In some embodiments, where the methods refer to an aldehyde trapping agent, the aldehyde trapping agent is reproxalap. In some embodiments, the reproxalap is in a composition for topical administration to the eye, particularly an ophthalmic aqueous solution. In some embodiments, reproxalap is at a concentration of 0.1% to 0.5% w/v. In some embodiments, the reproxalap is at a concentration of 0.15% to 0.45% w/v, 0.2% to 0.4% w/v. In some embodiments the reproxalap is at a concentration of 0.1% w/v, 0.15% w/v, 0.2% w/v, 0.25% w/v, 0.3% w/v, 0.35% w/v, 0.4% w/v, 0.45% w/v, 0.5% w/v. In some embodiments, the reproxalap is at a concentration of 0.25% w/v. In some embodiments, the reproxalap is at a concentration of 0.1% w/v. In some embodiments, the reproxalap is at a concentration of 0.5% w/v.

In some embodiments, the reproxalap is in an admixture with a pharmaceutically acceptable excipient, wherein the excipient is a cyclodextrin selected from sulfobutylether-p-cyclodextrin and hydroxypropyl-β-cyclodextrin, or a pharmaceutically acceptable salt thereof. Preferably, the pharmaceutically acceptable excipient is sulfobutylether-β-cyclodextrin or a pharmaceutically acceptable salt thereof. In some embodiments, the cyclodextrin is present at 5% to 20% w/v, for example 6% to 15% w/v. In some embodiments, the cyclodextrin is present at about 7% w/v, 8% w/v, 9% w/v, 10% w/v, or 11% w/v. In some embodiments, the cyclodextrin is present at 7% w/v. In some embodiments, the cyclodextrin is present at 11% w/v.

In some embodiments, the reproxalap referenced in the methods is 0.25% w/v reproxalap and 7% w/v of cyclodextrin, particularly sulfobutylether-β-cyclodextrin. In some embodiments, the reproxalap referenced in the methods is 0.25% w/v reproxalap and 11% w/v of cyclodextrin, particularly sulfobutylether-β-cyclodextrin.

In another aspect, the present disclosure provides a kit for use in treating ocular inflammation in a subject, comprising: a container comprising an ophthalmic formulation comprising reproxalap as described herein; an assay kit for testing the levels of one or more aldehyde markers of ocular inflammation in the subject's tears as described herein; and, optionally, instructions for using the assay to test the levels of one or more aldehyde markers of ocular inflammation in the subject's tears.

### BRIEF DESCRIPTION OF THE DRAWINGS

FIG. 1 shows mean adduct concentrations in tears from D/C subjects collected at Visit 1 and normal human tears (NHT). Normal human tears diluted 20-fold had an average calculated MDA adduct concentration of 2,266 pmol/mL. Tears from D/C DES subjects had a mean MDA adduct concentration of 7,798 pmol/mL, a 3.4 fold increase in relative to NHT.

FIG. 2 shows mean MDA adduct concentrations in tears from 37 DES subject who completed the trial, at Visit 1 (baseline, before treatment) and Visit 3 (after 28 days of treatment with reproxalap ophthalmic solution. Tears collected at Visit 1 had a mean MDA adduct concentration of 14,943 pmol/mL, which is significantly higher than the mean MDA adduct concentration of 11,566 pmol/mL in tears collected from all subjects at Visit 3.

FIG. 3 shows mean MDA adduct concentrations in tears from 37 DES subject who completed the trial, at Visit 1 (baseline, before treatment) and Visit 3 (after 28 days of treatment with 0.1% w/v reproxalap ophthalmic solution. Tears from study subjects treated with reproxalap ophthalmic solution (0.1%) had a mean MDA adduct concentration of 14,287 pmol/mL at Visit 1, compared to 11,028 pmol/mL at Visit 3, which corresponds to a 23% reduction in MDA adduct levels after treatment.

FIG. 4 shows mean MDA adduct concentrations in tears from 37 DES subject who completed the trial, at Visit 1 (baseline, before treatment) and Visit 3 (after 28 days of treatment with 0.5% w/v reproxalap ophthalmic solution). DES study subjects showed a 26% reduction in MDA adduct concentration at Visit 3 compared to Visit 1.

FIG. 5 shows reduction in HNE-protein adduct levels in DED patient tears (run-in Phase 2/3 data, see Example 3). The data show Day 1 and Day 2 pre/post dose results (pre-dose to post-dose change) in HNE-protein adduct levels (pg/mL) in the patients dosed with either vehicle or reproxalap. Day 1 dose is first dose of Day 1. Day 2 dose is dose post chamber. Tear collections taken approximately 10 minutes before and after dosing.

FIG. 6 shows the mean of reduction in HNE-protein adduct levels in DED patient tears (run-in Phase 2/3 data, see Example 3). P values by group represent difference from 0 (no change). Means represent average of the two doses where tear RASP were assessed before and after dosing. HNE=4-hydroxynonenal ELISA of protein adducts. Tear RASP levels from the Phase 3 clinical trial run-in cohort were reduced after single doses of the novel RASP inhibitor reproxalap, as assessed by enzyme-linked immunosorbent assay (ELISA) of 4-hydroxynonenal protein adducts (HNE), a RASP selected based on results from a natural history study of dry eye patients. For subjects with sufficient tear volumes for analysis, across the two doses where tear RASP levels were assessed before and after drug administration, HNE levels declined by an average of 1018 picograms/milliliter (pg/mL) in reproxalap-treated patients (n=9) versus an increase of 32 pg/mL in vehicle-treated patients (n=7).

FIG. 7 shows VAS ocular dryness score results from part 1 of a Phase 3 clinical trial of 0.25% reproxalap vs. vehicle over the course of a 12-week chronic treatment. Notably, reproxalap demonstrated statistically significant symptom improvements (for multiple symptoms) over vehicle as early as one week after treatment initiation. Treatment Difference of induction-maintenance dosing, defined as the difference between the changes from baseline for the evaluated drug vs. vehicle (LS Mean Difference ±95% CI). Ocular Dryness Score co-primary endpoint assessed in pre-specified patient population having an OD4S dryness baseline score of ≥3 (N=170). Topical ocular reproxalap has been studied in over 1,100 patients with no observed safety concerns; mild instillation site irritation is the most commonly reported adverse event in clinical trials. Induction-Maintenance dosing defined as QID dosing (4× daily) for weeks 1-4 followed by BID dosing (2× daily) for weeks 5-8. Source: Reproxalap RENEW-Part 1 clinical trial results and TRANQUILITY Run-In Cohort initial results. VAS=Visual Analog Scale; OD4S=Ocular Discomfort & 4-Symptom; CAC=Conjunctival Allergen Challenge; MMRM=Mixed Effect Model Repeated Measures.

FIG. 8 shows MDA concentration in tears of pooled reproxalap groups. At baseline and after completion of treatment, MDA was measured in the tears of dry eye disease patients by ELISA (Cell Biolabs, San Diego, Calif.) in tears extracted through capillary. Both eyes were pooled per patient. A standard curve was generated, and a 1:60 dilution was established as optimal using 3 mL of tears per patient. Above- and below median percentage MDA reduction subgroups were compared using 2-way t tests and 1-way t tests versus 0 (no change from baseline). (A) Within-participant tear MDA adduct levels before treatment were compared with tear MDA adduct levels after treatment. (B) Total lissamine green staining scores at day 28 in participants with below median MDA adduct reduction after treatment were compared with those of participants with above-median MDA adduct reduction after treatment. MDA=malondialdehyde.

FIG. 9 shows Ocular Dryness Score (VAS) results of the TRANQUILITY Phase 3 Trial run-in cohort on Day 2. The results were obtained in a dry eye chamber and measured VAS dryness score over the course of 90 minutes in the chamber. Mixed effect model of repeated measures (MMRM) for change from baseline (Time 0). Topical ocular reproxalap has been studied in over 1,100 patients thus far with no observed safety concerns; mild instillation site irritation is the most commonly reported adverse event in clinical trials. VAS=Visual Analog Scale; MMRM=Mixed Model Repeated Measures.

FIG. 10 shows Ocular Discomfort Score results of the TRANQUILITY Phase 3 Trial run-in cohort. The results show that statistically significant drug effect over vehicle in ocular discomfort symptoms was observed upon entry and sustained throughout the duration of exposure. Like Ocular Dryness Score results, therapeutic activity was demonstrated after first and second doses, representing near-immediate (within minutes) and statistically significant symptom relief.

FIG. 11 shows Ocular Redness (Mean Ocular Redness Score 0-4) results of the TRANQUILITY Phase 3 Trial run-in cohort. The results show that statistically significant drug effect over vehicle in ocular discomfort symptoms was observed upon entry and sustained throughout the duration of exposure. Like Ocular Dryness Score results, therapeutic activity was demonstrated after first and second doses, representing near-immediate (within minutes) and statistically significant symptom relief Ocular redness is an FDA-approvable objective sign endpoint for dry eye disease. Currently FDA approved dry eye products have utilized Schirmer's Test.

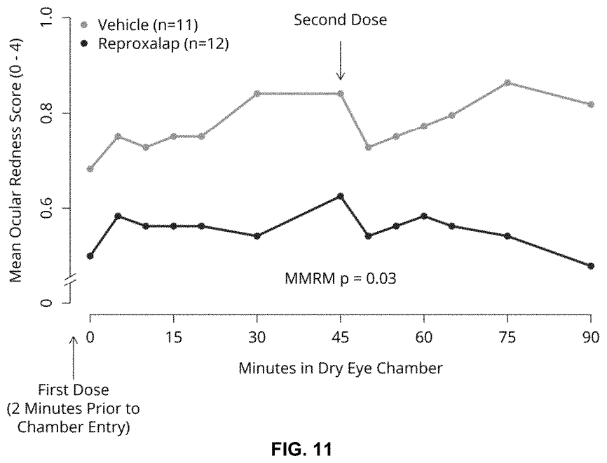

FIG. 12 shows (top) the initial results of a HABA/avidin/biotin assay for measuring levels of HNE-protein adducts in biological samples; and (bottom) the initial results of a streptavidin plate/lysozyme antibody assay for measuring levels of HNE-protein adducts in biological samples.

### DETAILED DESCRIPTION

### 1. General Description of Certain Embodiments

Reactive aldehyde species (RASP) are reactive organic molecules that bind to proteins, carbohydrates, lipids, and nucleic acids. RASP that are not sequestered or otherwise protected in specific metabolic processes are toxic, and aldehyde binding to cellular constituents leads to inflammation via activation of NFNB and other pro-inflammatory mediators, molecular dysfunction, and the accumulation of indigestible metabolites, such as lipofuscin components in the retina.

Reproxalap topical ocular solution is being developed for treatment of ocular inflammation, ocular dryness, ocular irritation, ocular redness, ocular itching, and other symptoms of ocular discomfort. Ocular discomfort symptoms include dryness, itchiness, tearing, burning, stinging, grittiness, foreign body sensation, cloudy vision, sensitivity to environment, sensitivity to light (photophobia), and stringy ocular secretion. In some embodiments, treatment of ocular symptoms is measured by an assay selected from ocular vital staining, tear film break-up time, tear osmolarity, and tear volume. In some embodiments, treatment of ocular symptoms is measured by an assay selected from Visual analog scale eye dryness score assessed over 24 hours after a first dose of reproxalap, and over 90 minutes in CAE® (Controlled Adverse Environment) Ora Calibra® Ocular Discomfort Scale assessed over 24 hours after first dose of reproxalap, and over 90 minutes in CAE® Ocular Discomfort & 4-Symptom Questionnaire assessed over 24 hours after first dose of reproxalap, and before and after CAE®; Ora Calibra® Conjunctival Allergen Challenge Ocular Itching Scale assessed over 24 hours after first dose of reproxalap, and before and after CAE®; Schirmer's Test change from baseline before and after the final dose of reproxalap; Change in tear RASP levels before and after a dose of reproxalap; Conjunctival Redness over 24 hours after first dose of reproxalap. The drug product, in various strengths, has completed a Phase 2a clinical trial and a controlled, double-masked Phase 2b clinical trial in dry eye disease.

The objective of the Phase 2a clinical trial in dry eye disease was to assess the safety, tolerability, and pharmacodynamic activity of Reproxalap Ophthalmic Solutions in subjects with dry eye disease (DED) for 28 days of QID dosing with one of three different formulations. The formulations used were 0.1% w/v Reproxalap Ophthalmic Solution, and 0.5% w/v Reproxalap Ophthalmic Solution, and 0.5% w/v Ophthalmic Lipid Solution. No serious adverse events (SAEs) were observed during the 28-day treatment with any of the three reproxalap formulations, and no clinically significant change in visual acuity (VA), intraocular pressure (IOP), slit lamp biomicroscopic findings, or undilated funduscopic findings were observed. Drop comfort was less well tolerated with the 0.5% w/v Reproxalap Ophthalmic Solution and Ophthalmic Lipid Solution than with the 0.1% w/v Reproxalap Ophthalmic Solution. Statistically significant efficacy in within-subject improvement was observed over a broad array of DED signs and symptoms assessed as exploratory pharmacodynamics endpoints.

In a Phase 2b clinical trial, the efficacy of Reproxalap Ophthalmic Solutions (0.25% and 0.1%) was evaluated in patients with dry eye disease (see the details in Example 1). Administration of Reproxalap Ophthalmic Solutions for 12 weeks resulted in statistically significant improvements in multiple clinical assessments, including tear quantity (Schirmer's Test), tear quality (Tear Film Breakup Time (TBUT) and tear osmolarity), and ocular surface staining. While this group of clinical assessments is used in assessing efficacy of treatment, desirable are biomarkers for dry eye disease that can be quantitated and used as a basis for assessing the efficacy of treatments for dry eye disease. The biomarkers can also be used to guide treatment regimen and select/identify patients for treatment of dry eye disease. Accordingly, the present disclosure provides aldehyde markers of oxidative stress for use as biomarkers. The present disclosure further provides assays for use in quantifying aldehyde markers of oxidative stress and for use in evaluating a patient response to treatment with an aldehyde trapping agent such as reproxalap.

These aldehyde markers include aldehydes such as formaldehyde, acetaldehyde, acrolein, glyoxal, methylglyoxal, hexadecanal, octadecanal, hexadecenal, succinic semi-aldehyde, malondialdehyde, 4-hydroxynonenal (4-HNE or HNE), 4-hydroxy-2E-hexenal, 4-hydroxy-2E,6Z-dodecadienal, retinaldehyde, leukotriene B4 aldehyde, and octadecenal, as particular aldehydes which form adducts and one or more of which are present in a patient's tear. In some embodiments, a useful aldehyde marker of oxidative stress for dry eye disease is malondialdehyde or 4-hydroxynonenal or their adducts, particularly adducts of malondialdehyde or 4-hydroxynonenal present in the tear of patients. Measuring the levels of the aldehyde markers of oxidative stress can be used to determine the efficacy of a drug for treating dry eye disease or used to guide treatment regimes, particularly for drugs acting as aldehyde trapping agents. These biomarkers can also be used to identify or select patients for treatment of dry eye disease, as further described herein.

### 2. Definitions

The general terms used herein are defined with the following meanings, unless explicitly stated otherwise.

The term “comprising” and “including” are used herein in their open-ended and non-limiting sense unless otherwise noted. It is to be further understood that where descriptions of various embodiments use the term “comprising” or “including,” those skilled in the art would understand that in some specific instances, an embodiment can be alternatively described using language “consisting essentially of” or “consisting of.”

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

The term “pharmaceutically acceptable” is defined herein to refer to those compounds, biologic agents, materials, compositions and/or dosage forms, which are, within the scope of sound medical judgment, suitable for contact with the tissues a subject e.g., a mammal or human, without excessive toxicity, irritation allergic response and other problem complications commensurate with a reasonable benefit/risk ratio.

The term “treating” or “treatment” as used herein comprises a treatment relieving, reducing or alleviating at least one symptom in a subject or affecting a delay of progression of a disease, condition and/or disorder. For example, treatment can be the diminishment of one or several signs or symptoms of a disorder or complete eradication of a disorder. Within the meaning of the present invention, the term “treat” also denotes to arrest, delay the onset (e.g., the period prior to clinical manifestation of a disease) and/or reduce the risk of developing or worsening a disease.

The term “subject” as used herein includes animals, such as mammals, e.g., humans, dogs, cows, horses, pigs, sheep, goats, cats, mice, rabbits, rats and transgenic non-human animals. In some embodiments, the subject is a human, also referred to as patient. In some embodiments, a subject means a subject or patient which has dry eye disease.

The term “about” or “approximately” shall have the meaning of within 10% of a given value or range. In some embodiments, the term “about” refers to within 10%, 9%, 8%, 7%, 6%, 5%, 4%, 3%, 2%, or 1% of a given value.

The term “w/v” as used herein refers to “gram/mL” (weight over volume), which is a concentration unit. For example, 7% w/v is equivalent to 70 mg/mL.

“Aldehyde trapping agent” or “aldehyde conjugating agent” refers to an agent that is reactive with an aldehyde, for example malondialdehyde and 4-hydroxynonenal, to form an adduct or conjugate between the aldehyde trapping agent and the aldehyde.

Reproxalap, also referred to as ADX-102, is of formula

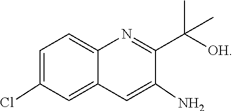 [SMILES: CC(C)(O)c1nc2ccc(Cl)cc2cc1N]

Without wishing to be bound by any particular theory, reproxalap functions as an aldehyde sequestering agent, or “trap,” which binds rapidly to aldehydes and forms a cyclic product.

### 3. Biomarkers of Dry Eye Disease and Uses Thereof

In one aspect, the present disclosure provides use of biomarkers for determining the efficacy of a treatment for dry eye disease, particularly a treatment with an aldehyde trapping agent. As noted above, aldehyde trapping agents comprise compounds that react with an aldehyde to form an adduct or conjugate between the aldehyde and the aldehyde trapping agent. In some embodiments, the aldehyde trapping agent is a chemical compound with an amino group capable of reacting with an aldehyde. In some embodiments, the aldehyde trapping agent has the general structure:

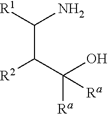

wherein R¹ and R² form a cycloalkyl, heterocycloalkyl, aryl, heteroaryl, or a fused bicyclic cycloalkyl, heterocycloalkyl, aryl, or heteroaryl ring; and each R^{a} is independently C₁₋₄ aliphatic optionally substituted with 1, 2, or 3 deuterium or halogen atoms; or each R^{a}, taken together with the carbon atom to which they are attached, form a 3- to 8-membered cycloalkyl or heterocyclyl ring containing 1-2 heteroatoms selected from nitrogen, oxygen, and sulfur. In some embodiments, each R^{a} is a straight chain alkyl, for example methyl, ethyl, propyl, or butyl, preferably methyl.

Various aldehyde trapping agents are disclosed in U.S. Pat. Nos. 7,973,025; 9,604,997; 9,814,701; 10,111,862; 9,687,481; 10,414,732; 10,550,085; U.S. patent publication 2018/0250306; U.S. patent publication 2018/0050989; U.S. patent publication 2020/0246345; U.S. patent publication 2020/0121591; and International patent publication WO2018039197; all of which are incorporated herein by reference. In some embodiments, an exemplary aldehyde trapping agent for the methods herein is reproxalap.

In some embodiments, the present disclosure provides a method of assessing effectiveness of an aldehyde trapping agent in treating dry eye disease in a patient. In some embodiments, a method of assessing effectiveness of an aldehyde trapping agent in treating dry eye disease in a patient comprises:

(i) administering an aldehyde trapping agent to a patient with dry eye disease;

(ii) measuring the level of an aldehyde marker of oxidative stress present in the eye of the patient; and

(iii) comparing the measured level of the aldehyde marker of oxidative stress to level of aldehyde marker of oxidative stress in an appropriate control;

wherein a reduction in level of aldehyde marker of oxidative stress indicates effectiveness of the aldehyde trapping agent in treating dry eye disease. In some embodiments, the initial administration of step (i) is a single administration of the aldehyde trapping agent. In some embodiments, the initial administration of step (i) is two, three, four, five, six, ten, or more doses of the aldehyde trapping agent; or is for a predetermined period of time, such as one, two, three, four, five, six, seven, or more days, or one week, two weeks, three weeks, one month, two months, or more. In some embodiments, the method further comprises the step of (iv) treating the dry eye disease by administering to the patient an effective amount of reproxalap if the patient exhibits in at least one of the patient's eyes a measured level of at least about 2 fold or greater of the aldehyde marker of oxidative stress relative to an appropriate control, such as a healthy human subject who does not have dry eye disease or any other ocular inflammation. In some embodiments, the treatment of dry eye disease is performed if a sufficient reduction in level of the aldehyde marker of oxidative stress is observed. Such sufficient reduction in level of the aldehyde marker of oxidative stress is selected from one of the parameters listed below. For example, in some embodiments an at least 15% or greater reduction in level of the aldehyde marker of oxidative stress compared to control level is indicative of effectiveness of the aldehyde trapping agent in treating dry eye disease.

If the level of the aldehyde marker is not reduced sufficiently, the dose of the aldehyde trapping agent and/or frequency of dosing may be increased. Alternatively, treatment is discontinued, and the patient is optionally treated with a different aldehyde trapping agent or a standard of care treatment for dry eye disease. In some embodiments, treatment is discontinued if a reduction of less than 10% in the level of the aldehyde marker of oxidative stress is obtained. In some embodiments, treatment is discontinued in a given patient or group of patients if the reduction in the level of the aldehyde marker of oxidative stress is below average; or more than one standard deviation worse (i.e., a smaller reduction) than the average.

In some embodiments, the treatment of dry eye disease is performed at an increased dose and/or dosing frequency of the aldehyde trapping agent relative to the initial administration of the aldehyde trapping agent to the patient with dry eye disease. In some embodiments, the treatment of dry eye disease comprises administering the aldehyde trapping agent an additional once, twice, thrice, or four times daily relative to the initial administration.

In some embodiments, as noted above, the aldehyde marker of oxidative stress in dry eye disease is formaldehyde, acetaldehyde, acrolein, glyoxal, methylglyoxal, hexadecanal, octadecanal, hexadecenal, succinic semi-aldehyde, malondialdehyde, 4-hydroxynonenal (4-HNE or HNE), 4-hydroxy-2E-hexenal, 4-hydroxy-2E,6Z-dodecadienal, retinaldehyde, leukotriene B4 aldehyde, or octadecenal. Particularly useful aldehyde markers of oxidative stress are those that form stable adducts, such as with proteins and other biomolecules. More preferably, the aldehyde markers of oxidative stress are those whose adducts can be detected in tears of a patient.

In some embodiments, the aldehyde marker of oxidative stress in dry eye disease is malondialdehyde or 4-hydroxynonenal. In some embodiments, the aldehyde marker of oxidative stress in dry eye disease is malondialdehyde.

In some embodiments, the measuring of the level of an aldehyde marker of oxidative stress is conducted on a sample of tears obtained from the patient, for example prior to and following treatment with an aldehyde trapping agent.

In some embodiments, an appropriate control for comparing the measured level of the aldehyde marker of oxidative stress in the eye of a patient is the level of the aldehyde marker of oxidative stress prior to administration of the aldehyde trapping agent or level of the aldehyde marker of oxidative stress in patients diagnosed with dry eye disease.

In some embodiments, the level of the aldehyde marker of oxidative stress measured is in the form of adducts of the aldehyde marker of oxidative stress present in the eye, particularly in the tear of the patient or a sample of tear obtained from the patient. In some embodiments, the adducts comprise stable adducts formed with biomolecules in the eye, such as nucleic acids and proteins, in particular adducts formed with proteins. In some embodiments, the adducts measured or detected comprise adducts formed with malondialdehyde. In some embodiments, the adducts measured or detected comprise adducts formed with 4-hydroxynonenal. These adducts can be detected and measured by various methods available in the art, as further discussed below.

In some embodiments of the method for assessing efficacy of treatment, at least 15% or greater reduction in level of the aldehyde marker of oxidative stress compared to control level is indicative of effectiveness of the aldehyde trapping agent in treating dry eye disease. In some embodiments, at least about 20% or greater reduction in level of the aldehyde marker of oxidative stress compared to control level is indicative of effectiveness of the aldehyde trapping agent in treating dry eye disease. In some embodiments, at least about 25% or greater reduction in level of the aldehyde marker of oxidative stress compared to control level is indicative of effectiveness of the aldehyde trapping agent in treating dry eye disease.

In some embodiments, an about 10-40% reduction in level of the aldehyde marker of oxidative stress compared to control level is indicative of effectiveness of the aldehyde trapping agent in treating dry eye disease. In some embodiments, an about 15-30% reduction in level of the aldehyde marker of oxidative stress compared to control level is indicative of effectiveness of the aldehyde trapping agent in treating dry eye disease. In some embodiments, an about 20-30% reduction in level of the aldehyde marker of oxidative stress compared to control level is indicative of effectiveness of the aldehyde trapping agent in treating dry eye disease. In some embodiments, an about 14-28%, or a 14-26%, 14-25%, 14-24%, 14-23%, 14-22%, 14-21%, 14-20%, 15-30%, 15-28%, 15-26%, 15-25%, 15-24%, 15-23%, 15-22%, 15-21%, 15-20%, 16-30%, 16-28%, 16-26%, 16-25%, 16-24%, 16-23%, 16-22%, 16-21%, 16-20%, 17-30%, 17-28%, 17-26%, 17-25%, 17-24%, 17-23%, 17-22%, 17-21%, 17-20%, 18-35%, 18-33%, 18-31%, 18-30%, 18-28%, 18-26%, 18-22%, 19-35%, 19-33%, 19-31%, 19-30%, 19-28%, 19-26%, 19-22%, 20-35%, 20-33%, 20-31%, 20-30%, 20-28%, 20-26%, 20-24%, 21-35%, 21-33%, 21-31%, 21-30%, 21-28%, 21-26%, 21-24%, 22-35%, 22-33%, 22-31%, 22-30%, 22-28%, 22-26%, 22-24%, 23-35%, 23-33%, 23-31%, 23-30%, 23-28%, 23-26%, 24-35%, 24-33%, 24-31%, 24-30%, 24-28%, 24-26%, 25-40%, 25-35%, 25-33%, 25-31%, 25-30%, 25-28%, or 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% reduction in level of the aldehyde marker of oxidative stress compared to control level is indicative of effectiveness of the aldehyde trapping agent in treating dry eye disease.

In some embodiments, the control level is a mean malondialdehyde adduct concentration of about 14,000 pmol/L to about 14,900 pmol/L as measured according to Example 2 in the tear of subjects with dry eye disease.

In some embodiments, a measured level of malondialdehyde adduct concentration of about 12,000 pmol/L or lower by an appropriate assay, such as that described in Example 2, is indicative of effectiveness of the aldehyde trapping agent in treating dry eye disease. In some embodiments, a measured level of malondialdehyde adduct concentration of about 11,500 pmol/L or lower is indicative of effectiveness of the aldehyde trapping agent in treating dry eye disease. In some embodiments, a measured level of malondialdehyde adduct concentration of about 11,000 pmoUL or lower is indicative of effectiveness of the aldehyde trapping agent in treating dry eye disease. In some embodiments, a measured level of malondialdehyde adduct concentration of about 10,500 pmol/L or lower is indicative of effectiveness of the aldehyde trapping agent in treating dry eye disease. In some embodiments, a measured level of malondialdehyde adduct concentration of about 10,000 pmol/L or lower is indicative of effectiveness of the aldehyde trapping agent in treating dry eye disease. In some embodiments, a measured level of malondialdehyde adduct concentration of about 9,500 pmol/L or lower is indicative of effectiveness of the aldehyde trapping agent in treating dry eye disease. In some embodiments, a measured level of malondialdehyde adduct concentration of about 12,000 pmol/L or lower as measured according to Example 2 in human tears is indicative of effectiveness of the aldehyde trapping agent in treating dry eye disease.

In some embodiments, a decrease in HNE levels in a patient's eye of at least about 500 picograms/milliliter (pg/mL) is indicative of effectiveness of the aldehyde trapping agent in treating dry eye disease. In some embodiments, a decrease in HNE levels of at least about 600, 700, 750, 800, 850, 900, 950, 1000, 1050, 1100, 1150, 1200, 1250, 1300, 1350, 1400, 1450, or 1500 pg/mL is indicative of effectiveness of the aldehyde trapping agent in treating dry eye disease. In some embodiments, a decrease in HNE levels of about 500-1500 pg/mL is indicative of effectiveness of the aldehyde trapping agent in treating dry eye disease. In some embodiments, a decrease in HNE levels of about 600-1450, 650-1400, 700-1350, 750-1300, 800-1250, 850-1200, 900-1150, 950-1100, or 1000-1050 pg/mL is indicative of effectiveness of the aldehyde trapping agent in treating dry eye disease. In some embodiments, a decrease in HNE levels of about 1018 pg/mL is indicative of effectiveness of the aldehyde trapping agent in treating dry eye disease. HNE levels may be measured by an appropriate assay, such as a modified version of that described in Example 2. In some embodiments, HNE levels are measured in the patient's tears. In some embodiments, HNE levels are measured by quantifying the concentration of an adduct of HNE with a protein.

In some embodiments, a measured level of malondialdehyde adduct concentration of about 12,000 pmol/L or lower (or another measured level recited above) and a decrease in HNE levels of about 600-1450, 650-1400, 700-1350, 750-1300, 800-1250, 850-1200, 900-1150, 950-1100, or 1000-1050 pg/mL is indicative of effectiveness of the aldehyde trapping agent in treating dry eye disease.

In some embodiments, the method is used to assess the effectiveness of aldehyde trapping compound reproxalap. In some embodiments, reproxalap is administered topically, such as in an ophthalmic aqueous solution. In some embodiments, reproxalap is administered in any form and concentration and dosing regimen described below.

One aspect of the present invention relates to the surprising discovery that, upon administration of an aldehyde trap such as reproxalap, specific (quantified) decreases in levels of aldehyde markers of oxidative stress, such as MDA and/or HNE, correlate with statistically significant improvement in one or more symptoms of dry eye disease in patients. By using the quantified decreases in levels of aldehyde markers of oxidative stress as biomarkers, it is now possible to provide more effective treatments for dry eye disease, monitor the effectiveness of reproxalap and other dry eye disease treatments, and select patients for treatment of dry eye disease, as described herein.

In some embodiments of the method, reproxalap is administered topically at a concentration of 0.1% to 0.5% w/v. In some embodiments, reproxalap is administered topically to the eye at a concentration of 0.25% w/v. In some embodiments, reproxalap is administered topically to the eye at a concentration of 0.1% w/v. In some embodiments, reproxalap is administered topically to the eye at a concentration of 0.5% w/v.

As further described herein, reproxalap is administered as an admixture with a pharmaceutically acceptable excipient, wherein the excipient is a cyclodextrin. In some embodiments, the cyclodextrin is selected from sulfobutylether-β-cyclodextrin and hydroxypropyl-β-cyclodextrin, preferably sulfobutylether-β-cyclodextrin. In some embodiments of the method, the cyclodextrin is present at 5% to 20% w/v. In some embodiments, the cyclodextrin is present at 7% to 11% w/v. In some embodiments, the cyclodextrin is present at 7% w/v. In some embodiments, the cyclodextrin is present at 11% w/v.

In some embodiments, reproxalap is at a concentration of 0.25% w/v and the cyclodextrin is present at 7% w/v, preferably sulfobutylether-β-cyclodextrin. In some embodiments, the reproxalap and cyclodextrin are present in the formulation at a ratio of reproxalap to sulfobutylether-β-cyclodextrin of about a mole of reproxalap per 3 moles of sulfobutylether-β-cyclodextrin.

In some embodiments, reproxalap is at a concentration of 0.25% w/v and the cyclodextrin is present at 11% w/v, preferably sulfobutylether-β-cyclodextrin. In some embodiments, the reproxalap and cyclodextrin is present in the formulation at a ratio of reproxalap to sulfobutylether-β-cyclodextrin of about a mole of reproxalap per 5 moles of sulfobutylether-β-cyclodextrin.

In some embodiments, the level of aldehyde marker of oxidative stress is measured after one or more of 6, 10, 14, 18, 24, or 28 days of treatment with the aldehyde trapping agent. In some embodiments, the level of the aldehyde marker of oxidative stress is measured after 28 days of treatment.

In some embodiments, various dosing regimens can be used in determining the effectiveness of the aldehyde trapping agent in treating dry eye disease. In some embodiments, the treatment comprises topically administering the aldehyde trapping agent up to six times a day. In some embodiments, the treatment comprises topically administering the aldehyde trapping agent four times a day (QID).

In some embodiments of assessing the effectiveness of treatment with an aldehyde trapping agent, the treatment comprises an initiation phase and/or exacerbation phase, followed by a maintenance phase, as further described herein. In some embodiments, the treatment in the initiation phase and/or exacerbation phase comprises topically administering the aldehyde trapping agent to the eye four times a day (QID), and the treatment in the maintenance phase comprises topically administering the aldehyde trapping agent to the eye 4 times a day (QID) or two times a day (BID).

In some embodiments where the aldehyde trapping agent is reproxalap, any of the treatment and dosing regimens described herein can be used.

In some embodiments of the method, measuring the levels of the aldehyde marker of oxidative stress is during and/or following the initiation and/or exacerbation phase. In some embodiments of the method, measuring the levels of the aldehyde marker of oxidative stress is in the maintenance phase, e.g., during the maintenance phase.

In another aspect, the present disclosure provides a method of treating dry eye disease in a patient using the biomarkers to guide the treatment regime, such as dosages and/or dosing frequency with an aldehyde trapping agent. In some embodiments, a method of treating dry eye disease in a patient comprises:
(i) measuring the level of an aldehyde marker of oxidative stress in the eye of a patient with dry eye disease prior to treatment;
(ii) treating the patient with an aldehyde trapping agent, wherein the aldehyde trapping agent is reproxalap and wherein the reproxalap is administered topically to the eye; and
(iii) measuring the level of the aldehyde marker of oxidative stress in the eye of the patient following treatment;
   wherein the patient is treated with a lower dosing frequency of reproxalap for a reduction of greater than about 20% in the measured level of the aldehyde marker of oxidative stress, and wherein the patient is treated with the same or higher dosing frequency of reproxalap for a reduction of about 20% or less in the measured level of the aldehyde marker of oxidative stress.

In some embodiments, as noted above, the aldehyde markers of oxidative stress in ocular inflammation are diverse and can include formaldehyde, acetaldehyde, acrolein, crotonaldehyde, glyoxal, methylglyoxal, pentanal, hexanal, hydroxyhexanal, hydroxyhexenal, hexadecanal, octadecanal, hexadecenal, succinic semi-aldehyde, malondialdehyde, 4-hydroxynonenal (4-HNE or HNE), 4-hydroxy-2E-hexenal, 4-hydroxy-2E,6Z-dodecadienal, retinaldehyde, leukotriene B4 aldehyde, malondialdehyde-acetaldehyde adducts (MAA) or octadecenal, and other aldehydes. Particularly useful aldehyde markers of oxidative are those that form stable adducts, such as with proteins and other biomolecules. More preferably, the aldehyde markers of oxidative stress are those whose adducts can be detected in tears of a patient.

In some embodiments, as noted above, the aldehyde marker of oxidative stress in dry eye disease is formaldehyde, acetaldehyde, acrolein, glyoxal, methylglyoxal, hexadecanal, octadecanal, hexadecenal, succinic semi-aldehyde, malondialdehyde, 4-hydroxynonenal (4-HNE or HNE), 4-hydroxy-2E-hexenal, 4-hydroxy-2E,6Z-dodecadienal, retinaldehyde, leukotriene B4 aldehyde, or octadecenal. Particularly useful aldehyde markers of oxidative stress are those that form stable adducts, such as with proteins and other biomolecules. More preferably, the aldehyde markers of oxidative stress are those whose adducts can be detected in tears of a patient.

In some embodiments, the aldehyde marker of oxidative stress in dry eye disease is malondialdehyde or 4-hydroxynonenal. In some embodiments, the aldehyde marker of oxidative stress in dry eye disease is malondialdehyde.

In some embodiments, the measuring of the level of an aldehyde marker of oxidative stress is conducted on a sample of tears obtained from the patient, for example prior to and following treatment with an aldehyde trapping agent.

In some embodiments, the level of the aldehyde marker of oxidative stress measured is in the form of adducts of the aldehyde marker of oxidative stress present in the eye, particularly in the tear of the patient or a sample of tear obtained from the patient. In some embodiments, the adducts comprise stable adducts formed with biomolecules in the eye, such as nucleic acids and proteins. In some embodiments, the adducts measured or detected comprise adducts formed with malondialdehyde. In some embodiments, the adducts measured or detected comprise adducts formed with 4-hydroxynonenal. These adducts can be detected and measured by various methods available in the art, as further discussed below.

In some embodiments of the method of treatment, reproxalap is administered topically at a concentration of 0.1% to 0.5% w/v. In some embodiments, reproxalap is administered topically to the eye at a concentration of 0.25% w/v. In some embodiments, reproxalap is administered topically to the eye at a concentration of 0.1% w/v. In some embodiments, reproxalap is administered topically to the eye at a concentration of 0.5% w/v.

As further described herein, reproxalap is administered as an admixture with a pharmaceutically acceptable excipient, wherein the excipient is a cyclodextrin. In some embodiments, the cyclodextrin is selected from sulfobutylether-β-cyclodextrin and hydroxypropyl-β-cyclodextrin, preferably sulfobutylether-β-cyclodextrin. In some embodiments of the method, the cyclodextrin is present at 5% to 20% w/v. In some embodiments, the cyclodextrin is present at 7% to 11% w/v. In some embodiments, the cyclodextrin is present at 7% w/v. In some embodiments, the cyclodextrin is present at 11% w/v.

In some embodiments, the reproxalap is at a concentration of 0.25% w/v and the cyclodextrin is present at 7% w/v, preferably sulfobutylether-β-cyclodextrin. In some embodiments, the reproxalap and cyclodextrin is present in the formulation at a ratio of reproxalap to sulfobutylether-β-cyclodextrin of about a mole of reproxalap per 3 moles of sulfobutylether-β-cyclodextrin.

In some embodiments, reproxalap is at a concentration of 0.25% w/v and the cyclodextrin is present at 11% w/v, preferably sulfobutylether-β-cyclodextrin. In some embodiments, the reproxalap and cyclodextrin is present in the formulation at a ratio of reproxalap to sulfobutylether-β-cyclodextrin of about a mole of reproxalap per 5 moles of sulfobutylether-β-cyclodextrin.

In some embodiments of the method of treatment, any of the dosages and dosing or treatment regimens described herein and below can be used for administering the reproxalap. In some embodiments, reproxalap is administered up to six times a day. In some embodiments, reproxalap is administered four times a day (QID).

In some embodiments of the method of treatment, the patient is treated with a lower dosing frequency of reproxalap for a reduction of about 25% or greater in the measured level of the aldehyde marker of oxidative stress.

In some embodiments of the method, the treatment for dry eye disease comprises an initiation and/or exacerbation phase, and a maintenance phase. In some embodiments, the measuring of the aldehyde markers of oxidative stress following treatment is done during the initiation phase. In some embodiments, the measuring is done during the exacerbation phase. In some embodiments, the measuring is done both during the initiation and the exacerbation phase. In some of these embodiments, reproxalap is administered four times a day in the initiation and/or exacerbation phase.

In some embodiments, the patient is treated with a lower dosing frequency of reproxalap in the maintenance phase for a reduction of greater than about 20% in the measured level of the aldehyde marker of oxidative stress in the initiation and/or exacerbation phase.

In some embodiments, the patient is treated with a lower dosing frequency of reproxalap in the maintenance phase for a reduction of about 25% or greater in the measured level of the aldehyde marker of oxidative stress in the initiation and/or exacerbation phase.

In some embodiments, the lower dosing frequency is two times a day (BID).

In another aspect, the present disclosure provides a method of selecting or identifying a patient for treatment of dry eye disease, particularly for treatments with aldehyde trapping agents. In some embodiments, a method of selecting a patient for treatment of dry eye disease comprises: measuring the level of an aldehyde marker of oxidative stress in an eye of a patient suspected of having dry eye disease, wherein a measured level of at least about 2 fold or greater level of the aldehyde marker of oxidative stress as compared to level of aldehyde marker of oxidative stress in patients without dry eye disease is indicated for treatment.

In some embodiments, the aldehyde marker of oxidative stress in dry eye disease is formaldehyde, acetaldehyde, acrolein, glyoxal, methylglyoxal, hexadecanal, octadecanal, hexadecenal, succinic semi-aldehyde, malondialdehyde, 4-hydroxynonenal (4-HNE or HNE), 4-hydroxy-2E-hexenal, 4-hydroxy-2E,6Z-dodecadienal, retinaldehyde, leukotriene B4 aldehyde, or octadecenal.

In some embodiments, the aldehyde marker of oxidative stress in dry eye disease is malondialdehyde or 4-hydroxynonenal. In some embodiments, the aldehyde marker of oxidative stress in dry eye disease is malondialdehyde.

In some embodiments, the measuring of the level of an aldehyde marker of oxidative stress is conducted on a sample of tears obtained from the patient, for example prior to and following treatment with an aldehyde trapping agent.

In some embodiments, the level of the aldehyde marker of oxidative stress measured is in the form of adducts of the aldehyde marker of oxidative stress present in the eye, particularly in the tear of the patient or a sample of tear obtained from the patient. In some embodiments, the adducts comprise stable adducts formed with biomolecules in the eye, such as nucleic acids and proteins. In some embodiments, the adducts measured or detected comprise adducts formed with malondialdehyde. In some embodiments, the adducts measured or detected comprise adducts formed with 4-hydroxynonenal. These adducts can be detected and measured by various methods available in the art, as further discussed below.

In some embodiments, the method is used to identify or select patients with dry eye disease for treatment with an aldehyde trapping agent. In some embodiments, the aldehyde trapping agent is reproxalap. In some embodiments, any of the ophthalmic formulations, dosages and dosing regimens, as well as various treatment regimens described herein is considered for treatment of the patient.

In some embodiments, the patient is selected or identified for treatment with reproxalap, wherein the reproxalap is administered topically at a concentration of 0.1% to 0.5% w/v. In some embodiments, the patient is selected or identified for treatment with reproxalap, wherein reproxalap is administered topically to the eye at a concentration of 0.25% w/v. In some embodiments, the patient is selected or identified for treatment with reproxalap, wherein reproxalap is administered topically to the eye at a concentration of 0.1% w/v. In some embodiments, the patient is selected or identified for treatment with reproxalap, wherein reproxalap is administered topically to the eye at a concentration of 0.5% w/v.

In some embodiments, the reproxalap is administered as an admixture with a pharmaceutically acceptable excipient, wherein the excipient is a cyclodextrin. In some embodiments, the cyclodextrin is selected from sulfobutylether-β-cyclodextrin and hydroxypropyl-β-cyclodextrin, preferably sulfobutylether-β-cyclodextrin. In some embodiments of the method, the cyclodextrin is present at 5% to 20% w/v. In some embodiments the cyclodextrin is present at 7% to 11% w/v.

In some embodiments, reproxalap is at a concentration of 0.25% w/v and the cyclodextrin is present at 7% w/v, preferably sulfobutylether-β-cyclodextrin. In some embodiments, the reproxalap and cyclodextrin is present in the formulation at a ratio of reproxalap to sulfobutylether-β-cyclodextrin of about a mole of reproxalap per 3 moles of sulfobutylether-β-cyclodextrin.

In some embodiments, reproxalap is at a concentration of 0.25% w/v and the cyclodextrin is present at 11% w/v, preferably sulfobutylether-β-cyclodextrin. In some embodiments, the reproxalap and cyclodextrin is present in the formulation at a ratio of reproxalap to sulfobutylether-β-cyclodextrin of about a mole of reproxalap per 5 moles of sulfobutylether-β-cyclodextrin.

In some embodiments for selecting or identifying a patient for treatment, the aldehyde marker of oxidative stress is in the form of malondialdehyde adduct, wherein a measured level malondialdehyde adduct of at least about 3.4 fold or greater as compared to level of malondialdehyde adduct in patients without dry eye disease is indicated for treatment of the patient.

In some embodiments herein, the level of aldehyde marker of oxidative stress and/or level of the adducts formed by the aldehyde markers of oxidative stress can be measured by any number of techniques. These include, by way of example and not limitation, mass spectroscopy (MS), chromatography (e.g., HPLC), LC/MS, antibody reagents (e.g., enzyme linked immunosorbent assay—ELISA). In some embodiments, the aldehyde markers of oxidative stress are detected by LC-MS, ultraviolet spectrometry (UV), HPLC, mass spectrometry (MS), monoclonal antibody detection assay, gas chromatography (GC), GC/MS, GC/flame ionization detector (FID), capillary electrophoresis with amperometric detection (CE-AD), liquid chromatography/fluorescence detection, or a combination thereof. These and other techniques are described in, for example, Houglum et al., J Clin Invest., 1990; 86(6): 1991-1998; Ishi et al., Chem. Res. Toxicol. 2006, 19(1):122-129; and Soares et al., J Liquid Chrom. & Related Technol., 2004; 27(15):2357-2369. In some embodiments, the assay is substantially as described in Example 2, Example 4, or Example 5, below. In some embodiments, the level of aldehyde marker of oxidative stress (e.g., MDA and/or HNE) and/or level of the adducts formed by the aldehyde marker of oxidative stress is/are used as an endpoint for a treatment regimen for ocular inflammation, such as dry eye disease.

### 4. Ophthalmic Solutions

In various embodiments referring to reproxalap and its use in the methods described above, the reproxalap, or a pharmaceutically acceptable salt thereof, is formulated as an ophthalmic solution at a concentration suitable for treating dry eye disease, in particular without causing severe or intolerable adverse effects. In some embodiments, any of the ophthalmic solutions described herein can be used in the methods. In some embodiments, the ophthalmic solution comprises about 0.1% to 0.5% w/v reproxalap, or a pharmaceutically acceptable salt thereof, and a pharmaceutically acceptable excipient. In some embodiments, the excipient comprises a cyclodextrin, such as sulfobutylether β-cyclodextrin (SBECD) or hydroxypropyl β-cyclodextrin.

In some embodiments, an ophthalmic solution comprises reproxalap and a cyclodextrin excipient in a ratio of less than 1:2.1 on a mole:mole basis. In some embodiments, the ratio of reproxalap and cyclodextrin is about 1:2.1 to about 1:25 ratio on a mole:mole basis. In some embodiments, the ratio is about 1:2.2 to 1:20, 1:2.5 to 1:20, 1:2.5 to 1:10, 1:2.75 to 1:10, 1:3 to 1:8, 1:3.5 to 1:7, 1:4 to 1:6, or 1:4 to 1:5 in a mole:mole basis. In some embodiments, the ratio is about 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1:3.6, 1:3.7, 1:3.8, 1:3.9, 1:4.0, 1:4.1, 1:4.2, 1:4.3, 1:4.4, 1:4.5, 1:4.6, 1:4.7, 1:4.8, 1:4.9, 1:5.0, 1:5.1, 1:5.2, 1:5.3, 1:5.4, 1:5.5, 1:6, 1:7, 1:8, 1:9, 1:10, 1:12, 1:15, 1:20, or 1:25 on a mole:mole basis.

In some embodiments, the cyclodextrin excipient is one of those described herein, such as sulfobutylether β-cyclodextrin (SBECD). The average degree of substitution of the SBECD is about 6.5.

In some embodiments, the ratio of reproxalap to the excipient is about 1:2.1 or less on a mole:mole basis.

In some embodiments, the excipient is a cyclodextrin and the ratio of reproxalap to the excipient is about 1:2.1 to about 1:25 on a mole:mole basis.

In some embodiments, the excipient is a cyclodextrin and the ratio of reproxalap to the excipient is about 1:2 to about 1:5 on a mole:mole basis.

In some embodiments, the present invention provides an ophthalmic solution comprising reproxalap, or a pharmaceutically acceptable salt thereof, and a pharmaceutically acceptable excipient, wherein the concentration of reproxalap, or a pharmaceutically acceptable salt thereof, is about 0.5% w/v or less and about 0.1% w/v or greater. In some embodiments, the ophthalmic solution comprises about 0.15 to about 0.45% w/v reproxalap, or a pharmaceutically acceptable salt thereof, and a pharmaceutically acceptable excipient. In some embodiments, the ophthalmic solution comprises about 0.2 to about 0.4% w/v reproxalap, or a pharmaceutically acceptable salt thereof, and a pharmaceutically acceptable excipient. In some embodiments, the ophthalmic solution comprises about 0.21 to about 0.35% w/v reproxalap, or a pharmaceutically acceptable salt thereof, and a pharmaceutically acceptable excipient. In some embodiments, the ophthalmic solution comprises about 0.22 to about 0.3% w/v reproxalap, or a pharmaceutically acceptable salt thereof, and a pharmaceutically acceptable excipient. In some embodiments, the ophthalmic solution comprises about 0.22 to about 0.29% w/v reproxalap, or a pharmaceutically acceptable salt thereof, and a pharmaceutically acceptable excipient. In some embodiments, the ophthalmic solution comprises about 0.25% w/v reproxalap, or a pharmaceutically acceptable salt thereof, and a pharmaceutically acceptable excipient. In some embodiments, the ophthalmic solution comprises about 0.25% w/v reproxalap and a pharmaceutically acceptable excipient selected from a cyclodextrin. In some embodiments, the ophthalmic solution comprises about 0.5% w/v reproxalap and a pharmaceutically acceptable excipient selected from a cyclodextrin.

In some embodiments, the present invention provides an ophthalmic solution comprising less than 0.5% w/v reproxalap, or a pharmaceutically acceptable salt thereof, and a pharmaceutically acceptable excipient. In some embodiments, the present invention provides an ophthalmic solution comprising at least 0.1% w/v reproxalap, or a pharmaceutically acceptable salt thereof, and a pharmaceutically acceptable excipient. In some embodiments, the present invention provides an ophthalmic solution comprising reproxalap, or a pharmaceutically acceptable salt thereof, and a pharmaceutically acceptable excipient, wherein the concentration of reproxalap, or a pharmaceutically acceptable salt thereof, is less than 0.5% w/v and 0.1% w/v or greater.

In some embodiments, reproxalap, or a pharmaceutically acceptable salt thereof, in an ophthalmic solution of the invention is at a concentration of less than 0.45% w/v and at least 0.1% w/v. In some embodiments, reproxalap, or a pharmaceutically acceptable salt thereof, in an ophthalmic solution of the invention is at a concentration of less than 0.4% w/v and at least 0.1% w/v. In some embodiments, reproxalap, or a pharmaceutically acceptable salt thereof, in an ophthalmic solution of the invention is at a concentration of less than 0.35% w/v and at least 0.1% w/v. In some embodiments, reproxalap, or a pharmaceutically acceptable salt thereof, in an ophthalmic solution of the invention is at a concentration of less than 0.3% w/v and at least 0.1% w/v. In some embodiments, reproxalap, or a pharmaceutically acceptable salt thereof, in an ophthalmic solution of the invention is at a concentration of less than 0.25% w/v and more than 0.1% w/v. In some embodiments, reproxalap, or a pharmaceutically acceptable salt thereof, in an ophthalmic solution of the invention is at a concentration of less than 0.2% w/v and at least 0.1% w/v. In some embodiments, reproxalap, or a pharmaceutically acceptable salt thereof, in an ophthalmic solution of the invention is at a concentration of less than 0.15% w/v and at least 0.1% w/v.

In some embodiments, reproxalap, or a pharmaceutically acceptable salt thereof, in an ophthalmic solution of the invention is at a concentration of 0.5% w/v or less and at least 0.15% w/v. In some embodiments, reproxalap, or a pharmaceutically acceptable salt thereof, in an ophthalmic solution of the invention is at a concentration of 0.5% w/v or less and at least 0.2% w/v. In some embodiments, reproxalap, or a pharmaceutically acceptable salt thereof, in an ophthalmic solution of the invention is at a concentration of 0.5% w/v or less and at least 0.25% w/v. In some embodiments, reproxalap, or a pharmaceutically acceptable salt thereof, in an ophthalmic solution of the invention is at a concentration of 0.5% w/v or less and at least 0.3% w/v. In some embodiments, reproxalap, or a pharmaceutically acceptable salt thereof, in an ophthalmic solution of the invention is at a concentration of 0.5% w/v or less and at least 0.35% w/v. In some embodiments, reproxalap, or a pharmaceutically acceptable salt thereof, in an ophthalmic solution of the invention is at a concentration of 0.5% w/v or less and at least 0.4% w/v. In some embodiments, reproxalap, or a pharmaceutically acceptable salt thereof, in an ophthalmic solution of the invention is at a concentration of 0.5% w/v or less and at least 0.45% w/v.

In some embodiments, reproxalap, or a pharmaceutically acceptable salt thereof, in an ophthalmic solution of the invention is at a concentration of about 0.1% to 0.5%, 0.15% to 0.45% w/v, 0.15% to 0.4% w/v, 0.15% to 0.35% w/v, 0.15% to 0.3% w/v, 0.15% to 0.25% w/v, or 0.15% to 0.2% w/v. In some embodiments, reproxalap, or a pharmaceutically acceptable salt thereof, in an ophthalmic solution of the invention is at a concentration of 0.2% to 0.45% w/v, 0.2% to 0.4% w/v, 0.2% to 0.35% w/v, 0.2% to 0.3% w/v, or 0.2% to 0.25% w/v. In some embodiments, reproxalap, or a pharmaceutically acceptable salt thereof, in an ophthalmic solution of the invention is at a concentration of 0.25% to 0.45% w/v, 0.25% to 0.4% w/v, 0.25% to 0.35% w/v, or 0.25% to 0.3% w/v. In some embodiments, reproxalap, or a pharmaceutically acceptable salt thereof, in an ophthalmic solution of the invention is at a concentration of 0.3% to 0.45% w/v or 0.3% to 0.4% w/v.

In some embodiments, reproxalap, or a pharmaceutically acceptable salt thereof, in an ophthalmic solution of the invention is at a concentration of about 0.1% w/v, 0.15% w/v, about 0.2% w/v, about 0.25%, about 0.3% w/v, about 0.35% w/v, about 0.4% w/v, about 0.45% w/v, or about 0.5% w/v.

In some embodiments, as further described herein, the foregoing concentrations of reproxalap can be selected and applied to treatment regimen that includes an initiation phase, an exacerbation phase, and/or a maintenance phase.

In some embodiments, a pharmaceutically acceptable excipient in an ophthalmic solution of the invention is a cyclodextrin. In some embodiments, the cyclodextrin is α-, β- or γ-cyclodextrin. In some embodiments, a cyclodextrin is a pharmaceutically acceptable derivative of a cyclodextrin, including, but not limited to, the hydroxyalkyl derivatives of α-, β- and γ-cyclodextrin (especially the hydroxyethyl and hydroxypropyl derivatives of β-cyclodextrin and γ-cyclodextrin), randomly methylated β-cyclodextrin, sulfobutylether β-cyclodextrin, sulfobutylether γ-cyclodextrin, and the so-called branched β- and γ-cyclodextrin derivatives such as glucosyl-β-cyclodextrin and glucosyl-γ-cyclodextrin. The natural cyclodextrins are either used alone or in a mixture of two or more cyclodextrins, by way of non-limiting example, a mixture of the γ-cyclodextrin and the more water-soluble hydroxypropyl γ-cyclodextrin, or γ-cyclodextrin and sulfobutylether γ-cyclodextrin, or β-cyclodextrin and hydroxypropyl-β-cyclodextrin, or β-cyclodextrin and sulfobutylether β-cyclodextrin.

In some embodiments, a cyclodextrin in an ophthalmic solution of the invention is at a concentration of 0 to 20% w/v. In some embodiments, a cyclodextrin in an ophthalmic solution of the invention is at a concentration of 1 to 18% w/v, 1 to 16% w/v, 1 to 14% w/v, 2 to 12% w/v, 4 to 10% w/v, 5 to 9% w/v, or 6 to 8% w/v. In some embodiments, the cyclodextrin in an ophthalmic solution of the invention is at a concentration of 7% to 11% w/v. In some embodiments, a cyclodextrin in an ophthalmic solution of the invention is at a concentration of about 1% w/v, 2% w/v, 3% w/v, 4% w/v, 5% w/v, 6% w/v, 7% w/v, 8% w/v, 9% w/v, 10% w/v, 11% w/v, 12% w/v, 13% w/v, 14% w/v, 15% w/v, 16% w/v, 17% w/v, 18% w/v, 19% w/v, or 20% w/v.

In some embodiments, a pharmaceutically acceptable excipient in an ophthalmic solution of the invention is sulfobutylether-β-cyclodextrin, in particular at any of the specified concentrations and ranges of concentrations above, such as about 7% w/v. In some embodiments, a pharmaceutically acceptable excipient in an ophthalmic solution of the invention is hydroxypropyl-β-cyclodextrin, in particular at any of the specified concentrations and ranges of concentrations specified above, such as about 7% w/v.

In some embodiments, the ophthalmic solution comprises about 0.2% to 0.4% w/v reproxalap and about 7% to 25% w/v of a cyclodextrin excipient such as SBECD. In some embodiments, the ophthalmic solution comprises about 0.2%, 0.25%, 0.3%, 0.35%, or 0.4% w/v reproxalap and about 7% to 25% w/v of a cyclodextrin excipient such as SBECD.

In some embodiments, the ophthalmic solution comprises about 0.25% w/v reproxalap and about 4.7% to about 25% w/v of a cyclodextrin excipient such as SBECD.

In some embodiments, the ophthalmic solution comprises about 0.25% w/v reproxalap and about 7% to 25% w/v of a cyclodextrin excipient such as SBECD.

In some embodiments, the ophthalmic solution comprises about 0.25% w/v reproxalap and about 4.75% to about 11% w/v of a cyclodextrin excipient such as SBECD.

In some embodiments, the ophthalmic solution comprises about 0.5% w/v reproxalap and about 9.5% to about 11% w/v of a cyclodextrin excipient such as SBECD. In some embodiments, the ratio of reproxalap to SBECD is about a mole of reproxalap per 2 moles of SBECD.

In some embodiments, the ophthalmic solution comprises about 0.25% w/v reproxalap and about 7% w/v of a cyclodextrin excipient such as SBECD. In some embodiments, the ratio of reproxalap to SBECD is about a mole of reproxalap per 3 moles SBECD.

In some embodiments, the ophthalmic solution comprises about 0.25% w/v reproxalap and about 11% w/v of a cyclodextrin excipient such as SBECD. In some embodiments, the ratio of reproxalap to SBECD is about a mole of reproxalap per 5 moles SBECD.

In some embodiments, an ophthalmic solution of the invention comprises a pharmaceutically acceptable buffering agent. In some embodiments, a pharmaceutically acceptable buffering agent is a phosphate buffer, citrate buffer, tris buffer, histidine buffer or acetate buffer.

In some embodiments, a pharmaceutically acceptable buffering agent is sodium phosphate, dibasic. In some embodiments, a pharmaceutically acceptable buffering agent is sodium phosphate, monobasic. In some embodiments, a pharmaceutically acceptable buffering agent is a mixture of sodium phosphate, dibasic, and sodium phosphate, monobasic. In some embodiments, an ophthalmic solution of the invention comprises about 0.083% w/v sodium phosphate, dibasic, and about 0.017% w/v sodium phosphate, monobasic.

In some embodiments, the ophthalmic solution of the invention is at an approximately neutral pH. In some embodiments, an ophthalmic solution of the invention is at a pH of 6.5 to 8. In some embodiments, an ophthalmic solution of the invention is at a pH of 6.9 to 7.7. In some embodiments, an ophthalmic solution of the invention is at a pH of 7.1 to 7.5. In some embodiments, an ophthalmic solution of the invention is at a pH of about 7.3. In some embodiments, an ophthalmic solution of the invention is at a pH of 7.3±0.01.

Pharmaceutically acceptable acids and/or bases may be used in the ophthalmic solution to adjust pH. In some embodiments, an ophthalmic solution of the invention comprises a pharmaceutically acceptable acid. In some embodiments, an ophthalmic solution of the invention comprises a pharmaceutically acceptable base. In some embodiments, an ophthalmic solution of the invention comprises a pharmaceutically acceptable acid and base. In some embodiments, a pharmaceutically acceptable acid is hydrochloric acid. In some embodiments, pharmaceutically acceptable base is sodium hydroxide.

In some embodiments, an ophthalmic solution of the invention comprises a tonicity agent. In some embodiments, a tonicity agent is selected from the group consisting of dextrose, potassium chloride, propylene glycol, and sodium chloride. In some embodiments, an ophthalmic solution of the invention comprises a tonicity agent at a concentration of less than about 0.5% w/v. In some embodiments, an ophthalmic solution of the invention comprises a tonicity agent at a concentration of about 0.45%, 0.4%, 0.35%, 0.3%, 0.25%, 0.2%, 0.15%, or 0.1% w/v. In some embodiments, a tonicity agent is sodium chloride.

In some embodiments, the ophthalmic solution comprises reproxalap at the specified concentrations, cyclodextrin, phosphate, and sodium chloride. In some embodiments. In some embodiments, the ophthalmic solution comprises reproxalap at the specified concentrations herein (e.g., 0.1% w/v, 0.25% w/v, 0.5% w/v, etc.), 5 to 9% w/v cyclodextrin (e.g., sulfobutylether-β-cyclodextrin or hydroxypropyl-β-cyclodextrin); 0.07% to 0.09% w/v sodium phosphate (dibasic), 0.015% to 0.19% w/v sodium phosphate (monobasic), and 0.2 to 0.3% w/v sodium chloride. In some embodiments, the ophthalmic solution comprises reproxalap at the specified concentrations herein (e.g., 0.1% w/v, 0.25% w/v, 0.5% w/v, etc.), about 7% w/v cyclodextrin (e.g., sulfobutylether-β-cyclodextrin or hydroxypropyl-β-cyclodextrin); 0.07% to 0.09% w/v sodium phosphate (dibasic), 0.015% to 0.019% w/v sodium phosphate (monobasic), and 0.2 to 0.3% w/v sodium chloride. In some embodiments, the ophthalmic solution is adjusted to an appropriate pH with sodium hydroxide or HCl.

In some embodiments, the ophthalmic solution comprises the following (0.5% Reproxalap Ophthalmic Solution A):

| Component | Amount (% w/v) | Grade |
|---|---|---|
| ADX-102 (reproxalap) | 0.5% | GMP |
| Sulfobutylether-beta-cyclodextrin | 9.5% | USP |
| (SBECD) | | |
| Sodium phosphate, dibasic, anhydrous | 0.083% | USP |
| Sodium phosphate, monobasic, | 0.017% | USP |
| monohydrate | | |
| Sodium hydroxide or Hydrochloric acid | pH adjustment | USP/NF |
| Sterile Water for Injection (WFI) | Dilute to volume | USP |

In some embodiments, the ophthalmic solution comprises the following (0.5% Reproxalap Ophthalmic Solution B)

| Component | Amount (% w/v) | Grade |
|---|---|---|
| ADX-102 (reproxalap) | 0.5% | GMP |
| Sulfobutylether-beta-cyclodextrin | 9.5% | USP |
| (SBECD) | | |
| Sodium hydroxide or Hydrochloric acid | pH adjustment | USP/NF |
| Sterile Water for Injection (WFI) | Dilute to volume | USP |

In some embodiments, the ophthalmic solution comprises the following (0.25% Reproxalap Ophthalmic Solution A)

| Component | Amount (% w/v) | Grade |
|---|---|---|
| ADX-102 (reproxalap) | 0.25% | GMP |
| Sulfobutylether-beta-cyclodextrin | 7.0% | USP |
| (SBECD) | | |
| Sodium phosphate, dibasic, anhydrous | 0.083% | USP |
| Sodium phosphate, monobasic, | 0.017% | USP |
| monohydrate | | |
| Sodium chloride | 0.24% | USP |
| Sodium chloride | Tonicity adjustment | USP |
| Sodium hydroxide or Hydrochloric acid | pH adjustment | USP/NF |
| Sterile Water for Injection (WFI) | Dilute to volume | USP |

In some embodiments, the ophthalmic solution comprises the following (0.25% Reproxalap Ophthalmic Solution B)

| Component | Amount (% w/v) | Grade |
|---|---|---|
| ADX-102 (reproxalap) | 0.25% | GMP |
| Sulfobutylether-beta-cyclodextrin | 7.0% | USP |
| (SBECD) | | |
| Sodium chloride | Tonicity adjustment | USP |
| Sodium hydroxide or Hydrochloric acid | pH adjustment | USP/NF |
| Sterile Water for Injection (WFI) | Dilute to volume | USP |

In some embodiments, the ophthalmic solution comprises the following (0.1% Reproxalap Ophthalmic Solution A)

| Component | Amount (% w/v) | Grade |
|---|---|---|
| ADX-102 (reproxalap) | 0.1% | GMP |
| Sulfobutylether-beta-cyclodextrin | 1.9% | USP |
| (SBECD) | | |
| Sodium phosphate, dibasic, anhydrous | 0.083% | USP |
| Sodium phosphate, monobasic, | 0.017% | USP |
| monohydrate | | |
| Sodium chloride | 0.75% | USP |
| Sodium hydroxide or Hydrochloric acid | pH adjustment | USP/NF |
| Sterile Water for Injection (WFI) | Dilute to volume | USP |

It is to be understood that variations of the ophthalmic solutions within the scope of the disclosure may be prepared given the guidance provided herein.

### 5. Administration and Dosages

In one aspect, the present invention provides a method for treating dry eye disease in a subject, comprising topically administering to an eye of a subject in need thereof a therapeutically effective amount of an ophthalmic solution of the invention. In some embodiments, the concentration of reproxalap in the ophthalmic solution used in the method is as described above.

In some embodiments, an ophthalmic solution of the invention can be administered at different frequencies suitable for effectively treating dry eye disease, for example, without causing severe or intolerable adverse effects.

In some embodiments, an ophthalmic solution of the invention can be topically administered one to six times a day. In some embodiments, a method of the invention comprises topically administering an ophthalmic solution of the invention six times a day. In some embodiments, a method of the invention comprises topically administering an ophthalmic solution of the invention five times a day. In some embodiments, a method of the invention comprises topically administering an ophthalmic solution of the invention four times a day (QID). In some embodiments, a method of the invention comprises topically administering an ophthalmic solution of the invention three times a day (TID). In some embodiments, a method of the invention comprises topically administering an ophthalmic solution of the invention two times a day (BID). In some embodiments, a method of the invention comprises topically administering an ophthalmic solution of the invention once a day (QD). In some embodiments, a method of the invention comprises topically administering an ophthalmic solution of the invention as needed (PRN).

In some embodiments, a method of the invention comprises topically administering to an eye of a subject with dry eye disease a therapeutically effective amount of an ophthalmic solution of the invention six times a day, five times a day, four times a day (QID), three times a day (TID), two times a day (BID), once a day (QD), followed by administration as needed (PRN).

In some embodiments, a method of the invention comprises topically administering an ophthalmic solution of the invention at various strengths (for example, at different reproxalap concentrations and different administration frequencies, as described herein).

In some embodiments, a method of the invention comprises topically administering an ophthalmic solution comprising about 0.25% w/v reproxalap, or a pharmaceutically acceptable salt thereof, four times a day, three times a day, or two times a day.

In some embodiments, a method of the invention comprises topically administering an ophthalmic solution comprising about 0.30% w/v reproxalap, or a pharmaceutically acceptable salt thereof, four times a day, three times a day, or two times a day.

In some embodiments, a method of the invention comprises topically administering an ophthalmic solution comprising about 0.35% w/v reproxalap, or a pharmaceutically acceptable salt thereof, four times a day, three times a day, or two times a day.

In some embodiments, a method of the invention comprises topically administering an ophthalmic solution comprising about 0.4% w/v reproxalap, or a pharmaceutically acceptable salt thereof, four times a day, three times a day, or two times a day.

In some embodiments, a method of the invention comprises topically administering an ophthalmic solution comprising about 0.45% w/v reproxalap, or a pharmaceutically acceptable salt thereof, four times a day, three times a day, or two times a day.

In some embodiments, a method of the invention comprises topically administering an ophthalmic solution comprising about 0.5% w/v reproxalap, or a pharmaceutically acceptable salt thereof, four times a day, three times a day, or two times a day.

In some embodiments, a method of the invention comprises topically administering an ophthalmic solution comprising 0.3% to 0.4% w/v reproxalap, or a pharmaceutically acceptable salt thereof, four times a day, three times a day, or two times a day.

In some embodiments, a method of the invention comprises topically administering an ophthalmic solution comprising 0.2% to 0.3% w/v reproxalap, or a pharmaceutically acceptable salt thereof, four times a day, three times a day, or two times a day.

In some embodiments, a method of the invention comprises topically administering an ophthalmic solution comprising 0.2% to 0.4% w/v reproxalap, or a pharmaceutically acceptable salt thereof, four times a day, three times a day, or two times a day.

In some embodiments, a method of the invention comprises two or more phases, wherein an ophthalmic solution of the invention is topically administering at different strengths in different phases. In some embodiments, a method of the invention comprises an initiation phase and a maintenance phase, wherein the ophthalmic solution is topically administered at a higher strength in the initiation phase than in the maintenance phase. In some embodiments, a treatment cycle of a method of the invention comprising multiple phases, including an exacerbation phase during which signs and/or symptoms become worse.

In some embodiments, the method of the invention comprises two or more phases, wherein an ophthalmic solution of the invention is topically administering at different strengths in different phases. In some embodiments, a method of the invention comprises an initiation phase, wherein the ophthalmic solution is topically administered at a high strength in the initiation phase, at a low strength in the maintenance phase, and at a high strength during an exacerbation of disease signs and/or symptoms.

In some embodiments, an ophthalmic solution administered in an initiation phase comprises a higher concentration of reproxalap, or a pharmaceutically acceptable salt thereof, than an ophthalmic solution administered in a maintenance phase. In some embodiments, the ophthalmic solution administered in an initiation phase or an exacerbation phase and the ophthalmic solution administered in a maintenance phase, comprises reproxalap, or a pharmaceutically acceptable salt, at a concentration selected from the group consisting of about 0.5% w/v, 0.45% w/v, 0.4% w/v, 0.35% w/v, 0.3% w/v, 0.25% w/v, 0.2% w/v, 0.15% w/v, and 0.1% w/v.

In some embodiments, an ophthalmic solution of about 0.5% w/v reproxalap is administered in an initiation phase or exacerbation phase, and less than 0.5% w/v reproxalap administered in a maintenance phase. In some embodiments, an ophthalmic solution of about 0.4% w/v, 0.35% w/v, 0.3% w/v, 0.25% w/v, 0.2% w/v, 0.15% w/v or 0.1% w/v reproxalap is administered in the maintenance phase.

In some embodiments, an ophthalmic solution of about 0.5% w/v to about 0.4% reproxalap is administered in an initiation phase or exacerbation phase, and less than 0.4% w/v reproxalap administered in a maintenance phase. In some embodiments, an ophthalmic solution of about 0.35% w/v, 0.3% w/v, 0.25% w/v, 0.2% w/v, 0.15% w/v or 0.1% w/v reproxalap is administered in the maintenance phase.

In some embodiments, an ophthalmic solution of about 0.5% w/v to about 0.3% reproxalap is administered in an initiation phase or exacerbation phase, and less than 0.3% w/v reproxalap administered in a maintenance phase. In some embodiments, an ophthalmic solution of about 0.25% w/v, 0.2% w/v, 0.15% w/v or 0.1% w/v reproxalap is administered in the maintenance phase.

In some embodiments, an ophthalmic solution of about 0.4% w/v to about 0.3% reproxalap is administered in an initiation phase or exacerbation phase, and less than 0.3% w/v reproxalap administered in a maintenance phase. In some embodiments, an ophthalmic solution of about 0.25% w/v, 0.2% w/v, 0.15% w/v or 0.1% w/v reproxalap is administered in the maintenance phase.

In some embodiments, an ophthalmic solution of about 0.3% w/v to about 0.2% reproxalap (e.g., 0.3%, 0.25%, or 0.2% w/v) is administered in an initiation phase or exacerbation phase, and 0.25% w/v or less reproxalap administered in a maintenance phase. In some embodiments, an ophthalmic solution of about 0.25% w/v, 0.2% w/v, 0.15% w/v or 0.1% w/v reproxalap is administered in the maintenance phase.

In some embodiments, an ophthalmic solution of the invention is topically administered more frequently per day in an initiation phase and an exacerbation phase than in a maintenance phase. In some embodiments, an ophthalmic solution of the invention is topically administered five times a day in an initiation phase, followed by four, three, two, or one times a day in a maintenance phase. In some embodiments, an ophthalmic solution of the invention is topically administering four times a day in an initiation phase or exacerbation phase, followed by three, two, or one times a day in a maintenance phase. In some embodiments, an ophthalmic solution of the invention is topically administering three times a day in an initiation phase or exacerbation phase, followed by two or one times a day in a maintenance phase. In some embodiments, an ophthalmic solution of the invention is topically administering two times a day in an initiation phase or exacerbation phase, followed by once daily in a maintenance phase.

In some embodiments, an ophthalmic solution administered in an initiation phase or exacerbation phase is at a higher reproxalap concentration and higher administration frequency than an ophthalmic solution administered in a maintenance phase.

In some embodiments, the present invention provides a method for treating dry eye disease in a subject, comprising topically administering to the subject an ophthalmic solution comprising about 0.4% w/v reproxalap, or a pharmaceutically acceptable salt thereof, and a pharmaceutically acceptable excipient, wherein the ophthalmic solution is administered at a higher strength in an initiation phase or exacerbation phase followed by a lower strength in a maintenance phase, wherein each of the initiation phase, exacerbation phase, and maintenance phase is as described herein.

In some embodiments, a multiple phase treatment cycle can include an initiation phase or exacerbation phase of up to 12 weeks with an ophthalmic solution comprising about 0.5%, 0.4% or 0.35% w/v (e.g., 0.5% to 0.35% w/v) reproxalap, or a pharmaceutically acceptable salt thereof, is up to 12 weeks, followed by a maintenance phase. In some embodiments, an ophthalmic solution comprising about 0.5%, 0.4% or 0.35% w/v (e.g., 0.5% to 0.35% w/v) reproxalap, or a pharmaceutically acceptable salt thereof, is administered four times a day in an initiation phase or exacerbation phase followed by three, two, or one times a day in the maintenance phase. In some embodiments, an ophthalmic solution comprising about 0.5%, 0.4% or 0.35% w/v (e.g., 0.5% to 0.35% w/v) reproxalap, or a pharmaceutically acceptable salt thereof, is administered three times a day in an initiation phase or exacerbation phase followed by two or one times a day in the maintenance phase.

In some embodiments, an ophthalmic solution comprising about 0.4%, 0.35% or 0.3% w/v (e.g., 0.4% to 0.3% w/v) reproxalap, or a pharmaceutically acceptable salt thereof, is administered four times a day in an initiation phase or exacerbation phase followed by three, two, or one times a day in the maintenance phase. In some embodiments, an ophthalmic solution comprising about 0.4%, 0.35% or 0.3% w/v (e.g., 0.4% to 0.3% w/v) reproxalap, or a pharmaceutically acceptable salt thereof, is administered three times a day in an initiation phase or exacerbation phase followed by two or one times a day in the maintenance phase.

In some embodiments, an ophthalmic solution comprising about 0.3%, 0.25% or 0.2% w/v (e.g., 0.3% to 0.2% w/v) reproxalap, or a pharmaceutically acceptable salt thereof, is administered four times a day in an initiation phase or exacerbation phase followed by three, two, or one times a day in the maintenance phase. In some embodiments, an ophthalmic solution comprising about 0.3%, 0.25% or 0.2% w/v (e.g., 0.3% to 0.2% w/v) reproxalap, or a pharmaceutically acceptable salt thereof, is administered three times a day in an initiation phase or exacerbation phase followed by two or one times a day in the maintenance phase.

In some embodiments, the present invention provides a method for treating dry eye disease in a subject, comprising topically administering to the subject an ophthalmic solution comprising 0.35% to 0.45% w/v reproxalap, or a pharmaceutically acceptable salt thereof, and a pharmaceutically acceptable excipient, wherein the ophthalmic solution is administered at a higher strength in an initiation phase or exacerbation phase followed by a lower strength in a maintenance phase, wherein each of the initiation phase, exacerbation phase and maintenance phase is as described herein. In some embodiments, a multiple phase treatment cycle of an ophthalmic solution comprising 0.35% to 0.45% w/v reproxalap, or a pharmaceutically acceptable salt thereof, is up to 12 weeks. In some embodiments, an ophthalmic solution comprising 0.35% to 0.45% w/v reproxalap, or a pharmaceutically acceptable salt thereof, is administered four times a day in an initiation phase or exacerbation phase followed by three, two, or one times a day in maintenance phase. In some embodiments, an ophthalmic solution comprising 0.35% to 0.45% w/v reproxalap, or a pharmaceutically acceptable salt thereof, is administered three times a day in an initiation phase or exacerbation phase followed by two or one times a day in maintenance phase.

In some embodiments, an ophthalmic solution is administered QID for about 10 to 14 weeks, preferably about 12 weeks. In some embodiments, an ophthalmic solution is administration QID for about 2 to 6 weeks, preferably about 4 weeks followed by administration BID for about 6 to 10 weeks, preferably about 8 weeks. In some embodiments, the ophthalmic solution for the foregoing treatment regimen is 0.25% w/v reproxalap, or a pharmaceutically acceptable salt thereof, and about 7% w/v SBECD.

In some embodiments, an ophthalmic solution is administered QID for about 2 to 6 weeks, preferably about 4 weeks, followed by administration BID for about 6 to 10 weeks, preferably about 8 weeks. In some embodiments, the ophthalmic solution for the foregoing treatment regimen is 0.25% w/v reproxalap, or a pharmaceutically acceptable salt thereof, and about 11% w/v SBECD.

In some embodiments, the present invention provides a method for treating certain subjects with dry eye disease. In some embodiments, a subject with dry eye disease is 18 years or older. In some embodiments, a subject with dry eye disease has a history of dry eye for at least six months prior to receiving the treatment of the invention. In some embodiments, a subject with dry eye disease has a history of use or desire to use eye drops for dry eye symptoms within six months prior to receiving the treatment of the invention.

In some embodiments, the present invention provides a method for treating a subject with dry eye disease, in particular moderate-to-severe dry-eye disease, comprising identifying subjects satisfying one or more of the following criteria for at least one eye, prior to receiving the treatment of the invention (for example, a screening performed at about one and/or two weeks before receiving the treatment):
having a Schirmer's Test score of ≤10 mm and ≥1 mm;
having a tear film break-up time (TFBUT©)≤5 seconds;
having a corneal fluorescein staining score of ≥2 in at least one region (e.g., inferior, superior, or central);
having a sum corneal fluorescein staining score of ≥4 based on the sum of the inferior, superior, and central regions; and
having a total Lissamine green conjunctival score of ≥2 based on the sum of the temporal and nasal regions.

In some embodiments, a subject with dry eye disease is not a female patient who is pregnant, nursing, or planning a pregnancy. In some embodiments, a subject with dry eye disease has not previously used reproxalap ophthalmic solution.

In some embodiments, the present invention provides a method for treating a subject with dry eye disease comprising a screening to exclude subjects having one or more of the following conditions for at least one eye, prior to receiving the treatment of the invention:
having any clinically significant slit lamp findings that may include active blepharitis, meibomian gland dysfunction (MGD), lid margin inflammation, or active ocular allergies that may require therapeutic treatment;
having an ongoing ocular infection (bacterial, viral, or fungal), or active ocular inflammation;
having previously had laser-assisted in situ keratomileusis (LASIK) surgery within the last 12 months;
having any planned ocular and/or lid surgeries over the study period or any ocular surgery within six months; and
having a known allergy and/or sensitivity to an ophthalmic solution of the invention or its components.

As described herein, an ophthalmic solution of the invention can achieve an early onset of effect in subjects with dry eye disease. As used herein, an “early onset effect” refers to early efficacy (e.g., within 1 to 2 weeks of initiation of treatment—in initiation or exacerbation phase) in ameliorating symptoms of dry eye disease. In some embodiments, the “early onset effect” is for the same dose and frequency of administration in the initiation or exacerbation phase. Accordingly, in some embodiments, the present invention provides a method for treating a subject with dry eye disease comprising topically administering to the subject an ophthalmic solution of the invention, wherein the ophthalmic solution is administered at a dose strength which can achieve an early onset profile. In some embodiments, an early onset profile comprises early onset of effect for symptoms (e.g., ocular discomfort including dryness, itchiness, tearing, burning, stinging, grittiness, cloudy vision, sensitivity to environment, stringy ocular secretion). In some embodiments, an early onset profile comprises early onset of effect for signs (e.g., ocular vital staining, tear film break-up time, tear osmolarity, tear volume).

In some embodiments, a dose strength which can achieve an early onset of effect comprises topically administering an ophthalmic solution comprising reproxalap, or a pharmaceutically acceptable salt thereof, at a concentration as described herein. In some embodiments, a dose strength which can achieve an early onset of effect comprises topically administering an ophthalmic solution comprising reproxalap, or a pharmaceutically acceptable salt thereof, at a frequency at described herein. In some embodiments, a dose strength which can achieve an early onset of effect comprises topically administering an ophthalmic solution comprising reproxalap, or a pharmaceutically acceptable salt thereof, at a concentration and a frequency at described herein.

In some embodiments, a method of the invention can achieve an onset of effect in about two weeks. At different dose strengths (for example, different concentration and administering frequency), a method of the invention can achieve an onset in fewer than about two weeks. For example, in some embodiments, a method of the invention can achieve an onset in about 14, 13, 12, 11, ten, nine, or eight days. At a certain dose strength, a method of the invention can achieve an onset in about one week or less. In some embodiments, a method of the invention can achieve an onset in about seven, six, five, four, three, two, or one days. In some embodiments, the early onset is accompanied by a reduction in an aldehyde marker of oxidative stress, for example about a 15%-30% reduction in level of the aldehyde marker of oxidative stress compared to control level. In some embodiments, the reduction is by about 20%.

In some embodiments, the present invention provides a method for treating dry eye disease in a subject, comprising topically administering to the subject an ophthalmic solution comprising about 0.5% w/v reproxalap, or a pharmaceutically acceptable salt thereof, and a pharmaceutically acceptable excipient, wherein the ophthalmic solution is administered three, two, or one times a day. In some embodiments, an ophthalmic solution comprising about 0.5% w/v reproxalap, or a pharmaceutically acceptable salt thereof, is administered three times a day. In some embodiments, an ophthalmic solution comprising about 0.5% w/v reproxalap, or a pharmaceutically acceptable salt thereof, is administered two times a day. In some embodiments, an ophthalmic solution comprising about 0.5% w/v reproxalap, or a pharmaceutically acceptable salt thereof, is administered once daily.

In some embodiments, the present invention provides a method for treating dry eye disease in a subject, comprising topically administering to the subject an ophthalmic solution comprising about 0.5% w/v reproxalap, or a pharmaceutically acceptable salt thereof, and a pharmaceutically acceptable excipient, wherein the ophthalmic solution is administered at a higher strength in an initiation phase or exacerbation phase, followed by a lower strength in a maintenance phase, wherein each of the initiation phase, exacerbation phase, and maintenance phase is as described herein. In some embodiments, an ophthalmic solution comprising about 0.5% w/v reproxalap, or a pharmaceutically acceptable salt thereof, is administered four times a day in an initiation phase or exacerbation phase followed by three, two, or one times a day in a maintenance phase. In some embodiments, an ophthalmic solution comprising about 0.5% w/v reproxalap, or a pharmaceutically acceptable salt thereof, is administered three times a day in an initiation phase or exacerbation phase followed by two or one times a day in a maintenance phase. In some embodiments, an ophthalmic solution comprising about 0.5% w/v reproxalap, or a pharmaceutically acceptable salt thereof, is administered two times a day in an initiation phase followed by one time a day in a maintenance phase. In some embodiments, an ophthalmic solution comprising about 0.5% w/v reproxalap, or a pharmaceutically acceptable salt thereof, is topically administered in an initiation phase or exacerbation phase, followed by topical administration of an ophthalmic solution comprising less than about 0.5% w/v reproxalap, or a pharmaceutically acceptable salt thereof, in a maintenance phase, wherein the administration frequency of each ophthalmic solution is selected from those as described above.

In another aspect, the present disclosure provides a kit or patent pack for use in treating ocular inflammation or a disease that causes ocular inflammation or other symptoms described above that produce elevated RASP levels in the eye of a subject. Such a kit comprises: a container comprising an ophthalmic formulation comprising reproxalap as described herein; an assay kit for testing the levels of one or more aldehyde markers of ocular inflammation in the subject's tears as described herein; and, optionally, instructions for using the assay to test the levels of one or more aldehyde markers of ocular inflammation in the subject's tears.

The formulation may be packaged in any suitable device or container, such as a flask, bottle, glass or plastic unit-dose container such as a bottle with an eye-dropper integrated into its lid, stick-pack, tube, ampoule, etc., typically under sterile conditions. More commonly the pharmaceutical formulations of the invention are prescribed to the patient in “patient packs” containing a number of dosing units or other means for administration of metered unit doses for use during a distinct treatment period in a single package. The inclusion of a package insert has been shown to improve patient compliance with the physician's instructions. The patient packs encompass at least one container containing the suitable amount of the liquid ophthalmic formulation as disclosed herein.

Alternatively, they can be supplied in a larger container as a bottle, then the invention also relates to pharmaceutical kit of parts comprising a bottle containing any liquid formulation as disclosed herein, a cap and/or an eye dropper or a dropper-cap system, and optionally instructions.

The bottle can be made of any material convenient with the storage and the use requirements comprising polymers, metal and glass and so on. It is of importance that the bottle material does not interfere with the components of the liquid formulation as disclosed herein. In an embodiment it is made of plastic.

### EXEMPLIFICATION

The following examples are intended to illustrate the invention and are not to be construed as being limitations thereon. Reproxalap can be synthesized as reported previously, for example, in WO 2006/127945, the entire content of which is incorporated herein by reference.

### Abbreviations

CAE: controlled adverse environment
GMP: Good Manufacturing Practice
ICH: International Council for Harmonization of Technical Requirements for
Pharmaceuticals for Human Use
OD: right eye
OS: left eye
OU: both eyes
PRN: as needed
QD: once daily
QID: Four times daily
QS: as much as will suffice

### Example 1: A Multi-Center, Phase 2b, Randomized, Double-Masked, Parallel-Group, Vehicle-Controlled, Clinical Study to Assess the Safety and Efficacy of Reproxalap Ophthalmic Solution (0.25% and 0.1%) Compared to Vehicle in Subjects with Dry Eye Disease

Objectives:
To evaluate the efficacy of Reproxalap Ophthalmic Solutions (0.25% and 0.1%) on baseline to weeks 2, 4, 8, and 12 change scores for sign and symptom endpoints of dry eye disease.
To evaluate effect sizes for efficacy endpoints of Reproxalap Ophthalmic Solutions (0.25% and 0.1%) vs vehicle for the treatment of the signs and symptoms of dry eye disease to confirm the endpoint selection and sample size for Phase 3 studies.
To evaluate the safety and tolerability of Reproxalap Ophthalmic Solutions (0.25% and 0.1%) to vehicle for the treatment of the signs and symptoms of dry eye disease.

Investigational Product:
1) Reproxalap Ophthalmic Solution (0.25%)
2) Reproxalap Ophthalmic Solution (0.1%)
3) Vehicle Ophthalmic Solution

In the Phase 2b study, reproxalap was formulated as an ophthalmic solution as described in the specification.

Duration: A subject's participation was estimated to be approximately 14 weeks (98 days).

Dosage/Dose Regimen/Instillation/Application/Use: Screening: Between Visits 1 and 2, all subjects received 14 consecutive days (±2) of Run-in (vehicle) ocular drops self-administered QID in both eyes.

Treatment: During the 12-week (84±3 days) treatment period, Reproxalap Ophthalmic Solution at concentrations of 0.1%, 0.25%, or vehicle ophthalmic solution was administered QID by bilateral topical ocular dosing. Subjects were randomized to one of three treatment groups (1:1:1) to receive study drug after the Post-CAE® assessments at Visit 2.

Summary of Visit Schedule: Six visits over the course of approximately 14 weeks
Visit 1=Day −14±2, CAE® Screening
Visit 2=Day 1, CAE® Confirmation/Baseline
Visit 3=Day 15±2, 2-Week Follow-Up
Visit 4=Day 29±2, 4-Week Follow-Up
Visit 5=Day 57±3, 8-Week Follow-Up
Visit 6=Day 85±3, 12-Week CAE® Follow-Up & Study Exit

Condition/Disease: Dry Eye Disease (DED)

Inclusion Criteria: Subjects for treatment were based on the following criteria:
1 Been at least 18 years of age of either gender and any race;
2 Provide written informed consent and sign the Health Information Portability and Accountability Act (HIPAA) form;
3 Had a reported history of dry eye for at least six months prior to Visit 1;
4 Had a history of use or desire to use eye drops for dry eye symptoms within six months of Visit 1;
5 Reported a score of ≥2 on the Ora Calibra® Ocular Discomfort & 4-Symptom Questionnaire in at least one symptom at Visit 1 and Visit 2 Pre-CAE®;
6 Had a Schirmer's Test score of ≤10 mm and >1 mm at Visit 1 and Visit 2;
7 Had a tear film break-up time (TFBUT©)≤5 seconds at Visit 1 and Visit 2 Pre-CAE®;
8 Had a corneal fluorescein staining score of ≥2 in at least one region (e.g., inferior, superior, or central) at Visit 1 and Visit 2 Pre-CAE®;
9 Have a sum corneal fluorescein staining score of ≥4, based on the sum of the inferior, superior, and central regions, at Visit 1 and Visit 2 Pre-CAE®;
10 Had a total Lissamine green conjunctival score of ≥2, based on the sum of the temporal and nasal regions at Visit 1 and Visit 2 Pre-CAE®;
11 Demonstrated a response to the CAE® at Visits 1 and 2 as defined by:
   A. Having at least a ≥1 point increase in fluorescein staining in the inferior region in at least one eye following CAE® exposure;
   B. Reporting an Ocular Discomfort score ≥3 at two or more consecutive time points in at least one eye during CAE® exposure (if a subject had an Ocular Discomfort rating of 3 at time=0 for an eye, s/he must have reported an Ocular Discomfort rating of 4 for two consecutive measurements for that eye). Note: a subject could not have an Ocular Discomfort score of 4 at time=0);
12 Had at least one eye, the same eye, satisfy all criteria for 6, 7, 8, 9, 10, and 11 above.

Exclusion Criteria: Subject were excluded based on the following criteria:
1 Had any clinically significant slit lamp findings at Visit 1 that may have included active blepharitis, meibomian gland dysfunction (MGD), lid margin inflammation, or active ocular allergies that require therapeutic treatment, and/or in the opinion of the investigator, might have interfered with study parameters;
2 Been diagnosed with an ongoing ocular infection (bacterial, viral, or fungal), or active ocular inflammation at Visit 1;
3 Worn contact lenses within seven days of Visit 1 or anticipate using contact lenses during the study;
4 Used any eye drops within 2 hours of Visit 1;
5 Had laser-assisted in situ keratomileusis (LASIK) surgery within the last 12 months;
6 Used cyclosporine 0.05% or lifitegrast 5.0% ophthalmic solution within 90 days of Visit 1;
7 Had any planned ocular and/or lid surgeries over the study period or any ocular surgery within 6 months of Visit 1;
8 Been using or anticipated using temporary punctal plugs during the study that had not been stable within 30 days of Visit 1;
9 Been currently taking any topical ophthalmic prescription (including medications for glaucoma) or over-the-counter (OTC) solutions, artificial tears, gels or scrubs, and cannot discontinue these medications for the duration of the trial (excluding medications allowed for the conduct of the study);
10 Had corrected visual acuity greater than or equal to logarithm of the minimum angle of resolution (log MAR)+0.7 as assessed by Early Treatment of Diabetic Retinopathy Study (ETDRS) scale in both eyes at Visit 1;
11 Been a woman who is pregnant, nursing, or planning a pregnancy;
12 Been unwilling to submit a urine pregnancy test at Visit 1 and Visit 6 (or early termination visit) if of childbearing potential. Non-childbearing potential was defined as a woman who is permanently sterilized (e.g., has had a hysterectomy or tubal ligation), or was postmenopausal (without menses for 12 consecutive months);
13 Been a man or woman of childbearing potential who was not using an acceptable means of birth control; acceptable methods of contraception include: hormonal—oral, implantable, injectable, or transdermal contraceptives; mechanical—spermicide in conjunction with a barrier such as a diaphragm or condom; intrauterine device (IUD); or surgical sterilization of partner. For non-sexually active males or females, abstinence may have been regarded as an adequate method of birth control; however, if the subject became sexually active during the study, he/she must have agreed to use adequate birth control as defined above for the remainder of the study;
14 Had a known allergy and/or sensitivity to the test article or its components;
15 Had a condition or be in a situation which the investigator feels may have put the subject at significant risk, confounded the study results, or interfered significantly with the subject's participation in the study;
16 Been currently enrolled in an investigational drug or device study or have used an investigational drug or device within 30 days of Visit 1;
17 Previously used reproxalap ophthalmic solution;
18 Been currently using any medication known to cause ocular drying that was not used on a stable dosing regimen for at least 30 days prior to Visit 1;
19 Been unable or unwilling to follow instructions, including participation in all study assessments and visits.

The following efficacy measures and endpoints were used in the study:
Lissamine green staining (Ora Calibra® scale); regions: inferior, superior, central, temporal, nasal, corneal sum, conjunctival sum, and total eye score)
Fluorescein staining (Ora Calibra® scale); regions: central, superior, inferior, temporal, nasal, corneal sum, conjunctival sum, and total eye score)
Tear film break-up time
Unanesthetized Schirmer's Test
Ora Calibra® Ocular Discomfort Scale
Ora Calibra® Ocular Discomfort & 4-Symptom Questionnaire
Ocular Surface Disease Index (OSDI)©
SANDE questionnaire
Tear Osmolarity

Safety Measures:
Visual acuity
Slit-lamp evaluation
Adverse event query
Intraocular Pressure (IOP)
Dilated fundoscopy

General Statistical Methods and Types of Analyses

Sample Size: The study sample size of 100 per group was selected based on prior Phase 2 and 3 clinical trial results using the DED Hybrid CAE study design with other development programs and the effect size seen in Phase 2a with reproxalap on change from baseline after four weeks of treatment. This sample size was deemed sufficient to assess the effect size on the DED sign and symptom endpoints with reproxalap vs vehicle, to confirm the endpoint selection and sample size needed for Phase 3 studies with reproxalap. A sample size of 100 per group provided 90% power at α=0.05 to detect an effect size of 0.26 for inferior Lissamine green staining (Ora Calibra® scale), assuming a common standard deviation of 0.56 and an effect size of 0.44 for ocular discomfort assessed with the Ora Calibra® Ocular Discomfort Scale assuming a common standard deviation of 0.97.

Efficacy Analysis
Evaluated baseline to weeks 2, 4, 8 and 12 change scores with reproxalap on DED sign and symptom endpoints (both pre-CAE and CAE endpoints). Each endpoint was analyzed at a two-sided alpha level of 0.05, and the overall type I error was not controlled for in this investigative study.
Evaluated effect size of baseline to weeks 2, 4, 8 and 12 change scores of reproxalap vs vehicle on DED sign and symptom endpoints (both pre-CAE and CAE endpoints) to confirm the endpoint selection for primary outcome parameters and sample size for Phase 3 studies with reproxalap.
Sub-group analyses on effect size of baseline to weeks 2, 4, 8 and 12 change scores of reproxalap vs vehicle on DED sign and symptom endpoints (both pre-CAE and CAE endpoints) [Subgroups were prospectively detailed in the Statistical Analysis Plan (SAP)].

TABLE 1
Summary of Subject Disposition

| | Reproxalap (0.1%) | | Reproxalap (0.25%) | | Vehicle | | All Subjects | |
|---|---|---|---|---|---|---|---|---|
| | N = 100 | | N = 100 | | N = 100 | | N = 300 | |
| Intent-to-Treat Population | 100 | (100.0%) | 100 | (100.0%) | 100 | (100.0%) | 300 | (100.0%) |
| Per Protocol Population | 97 | (97.0%) | 86 | (86.0%) | 98 | (98.0%) | 281 | (93.7%) |
| Safety Population | 100 | (100.0%) | 100 | (100.0%) | 100 | (100.0%) | 300 | (100.0%) |
| Study Completion | | | | | | | | |
| Completed | 97 | (97.0%) | 88 | (88.0%) | 99 | (99.0%) | 284 | (94.7%) |
| Discontinued | 3 | (3.0%) | 12 | (12.0%) | 1 | (1.0%) | 16 | (5.3%) |
| Reason for Study Withdrawal | | | | | | | | |

| | | | | | | | |
|---|---|---|---|---|---|---|---|
| Adverse Events | 2 | (2.0%) | 10 | (10.0%) | 0 | 12 | (4.0%) |

| | | | | | | |
|---|---|---|---|---|---|---|
| Administrative Reasons | 1 | (1.0%) | 0 | 0 | 1 | (0.3%) |

| | | | | | | | |
|---|---|---|---|---|---|---|---|
| Withdrawal by Subject | 0 | 1 | (1.0%) | 1 | (1.0%) | 2 | (0.7%) |

| | | | | | | |
|---|---|---|---|---|---|---|
| Other | 0 | 1 | (1.0%) | 0 | 1 | (0.3%) |

TABLE 2
Phase 2b AE Summary

| | Reproxalap 0.1% | | Reproxalap 0.25% | | Vehicle | | All Subjects | |
|---|---|---|---|---|---|---|---|---|
| | (N = 100) | | (N = 100) | | (N = 100) | | (N = 300) | |
| Number of Ocular TEAEs | 47 | | 111 | | 15 | | 173 | |
| Number of Subjects with Ocular TEAEs | 38 | (41.0%) | 93 | (93.0%) | 13 | (13.0%) | 144 | (48.0%) |
| Mild | 37 | (37.0%) | 84 | (84.0%) | 12 | (12.0%) | 133 | (44.3%) |
| Moderate | 1 | (1.0%) | 8 | (8.0%) | 1 | (1.0%) | 10 | (3.3%) |
| Severe | 0 | | 1 | (1.0%) | 0 | | 1 | (0.3%) |
| Instillation Site Pain Total | 37 | (37.0%) | 93 | (93.0%) | 2 | (2.0%) | 132 | (44.0%) |
| Prior to Day 15 | 31 | (31.0%) | 89 | (89.0%) | 1 | (1.0%) | 121 | (40.3%) |

| | | | | | | | |
|---|---|---|---|---|---|---|---|
| Day 15-Day 28 | 4 | (4.0%) | 4 | (4.0%) | 0 | 8 | (2.7%) |

| | | | | | | | |
|---|---|---|---|---|---|---|---|
| Day 29-Day 56 | 2 | (2.0%) | 0 | 1 | (1.0%) | 3 | (1.0%) |

| After Day 56 | 0 | 0 | 0 | 0 | |
|---|---|---|---|---|---|
| Number of Subjects with TEAEs | 1 | 10 | | | |
| leading to Discontinuation | | | | | |
| Prior to Day 15 | 1* | 7** | | | |
| Day 15-Day 28 | 0 | 2*** | 0 | 11 | (3.7%) |
| Day 29-Day 56 | 0 | 0 | | | |
| After Day 56 | 0 | 1 (SAE) | | | |
| Number of SAEs | 1 (peripheral | 1 (chest pain, | 0 | 2 | (0.7%) |
| | vertigo, not | not related) | | | |
| | related, stayed | | | | |
| | on study) | | | | |

*Subject discontinued at Day 12
**Subjects discontinued on the following Days: 2, 3, 5, 5, 5, 12, 14
***Subjects discontinued on the following Days: 15, 16

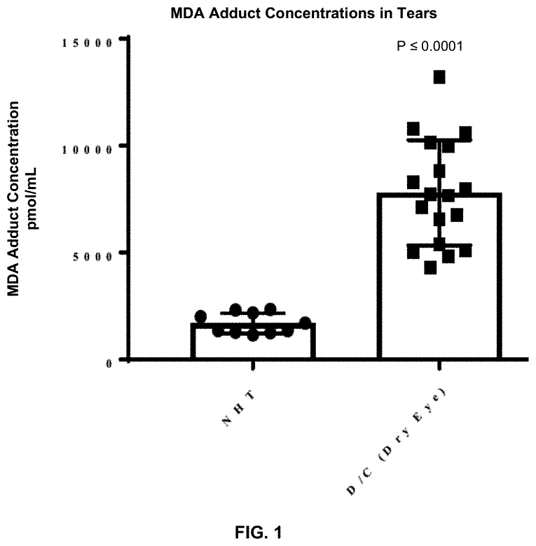

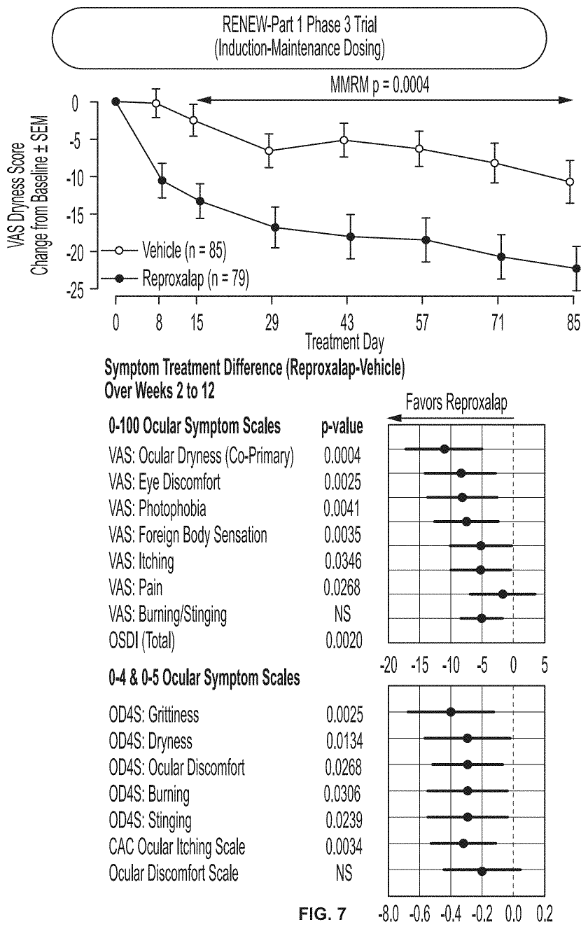

The phase 2b data are shown in FIGS. 1 through 9 and Tables 1 through 3.

Key Observations from Phase 2b Clinical Trial
1. Early onset of effect from Phase 2b evidenced across multiple signs and symptoms
   Majority (>50-100%) of effect vs vehicle seen at the first study endpoint (Week 2 or 4) in 0.25% group:
      Positive early onset for 3 out of 4 symptom endpoints: ODS, OD4SQ, OSDI
         Negative for SANDE
      Positive early onset for 3 out of 4 sign endpoints: Lissamine green total score, fluorescein total score, tear osmolarity
         Negative for TFBUT® (met definition at week 4)
         Schirmer's Test only assessed at week 12
2. Dose response was demonstrated between 0.1% and 0.25% dose strengths
3. 0.1% reproxalap matched higher dose effects at later time points
   Clearest effect with signs, especially ocular staining
   Compliance poorest in 0.25% group (8% non-compliant vs 3% in the 0.1% group and 1% in the vehicle group)
4. Vehicle effect increased with study duration
   Clearest effect was observed with signs, especially ocular staining
   Normal pattern in DED with plateau around two to three months
   QID vehicle in Phase 2b was expected to have increased this effect.

### Example 2: Measurement of Aldehyde Biomarkers in Tear Film in Patients in Clinical Trials

The study was to assess levels of malondialdehyde (MDA) adducts in tears collected from subjects with dry eye disease enrolled in a Phase 2a, randomized, double-masked, clinical study to assess the safety, tolerability, and pharmacodynamic activity of ADX-102 ophthalmic solution in subjects with dry eye diseases, also referred to herein as dry eye syndrome (DES).

Tears were collected during Visit 1, prior to treatment, and during Visit 3, following four weeks of treatment. Treatment groups consisted of ADX-102 Ophthalmic Solution (0.5%), ADX-102 Ophthalmic Solution (0.1%), and ADX-102 Ophthalmic Lipid Solution (0.5%).

Based on results from a prior study in which normal human tear (NHT) were diluted 1:20, resulting in readings in the linear range of a standard curve, 1:20 and 1:80 dilutions in D/C subject tear samples and NHT were tested to provide reference points for MDA adduct concentrations in tears from DES and normal subjects. In the prior study, the 1:80 dilution of the high concentration spiked control sample (20,000 pmol/mL) resulted in an OD value within the standard curve (data not shown). It was anticipated that tears from DES subjects would contain similar concentrations of MDA adducts, and therefore a 1:80 dilution would result in readings in the linear range of the standard curve.

The pilot assay was conducted using 1:20 and 1:80 dilution of samples, in duplicate. Data from the pilot study showed that OD values from the D/C subject tears diluted 1:20 and 1:80 were within the linear range of the standard curve.

The pilot assay was conducted using 1:20 and 1:80 dilution of samples, in duplicate. Based on the pilot assay data, the DES subject study samples were diluted 1:60 to maximize the likelihood of the OD values falling within the linear portion of the standard curve, and thus provide the most accurate results.

Human tears were collected from subjects with DES during the Phase 2a clinical trial, according to the schedule in the table below. Tears (up to 10 μL) from both eyes of each subject were collected and pooled at Visit 1. At Visit 3, after 28 days of treatment with ADX-102 Ophthalmic Solution, human tears were collected from both eyes of each subject still enrolled. The Visit 3 tear samples from both eyes of each subject were not pooled at the time of collection, which was not in accordance with the Tear Collection Procedure Manual. Instead, right eye and left eye samples from each subject were pooled at the time of analysis for MDA adducts. Out of the 51 enrolled subjects, 12 subjects dropped out before Visit 3 tear collection and were considered D/C subjects. Tears collected at Visit 1 from D/C subjects were used in MDA adduct assay method development.

| | Order of Events |
|---|---|
| Visit 1 | Informed Consent Demographics, Medical/Medication & Ocular |
| (Day 1): | History |
| Screening and | Pregnancy Testing |
| Enrollment | Acuity Slit Lamp Exam, Tear Osmolarity |
| | Tear Collection |
| | TFBUT |
| | Fluorescein and Lissamine Staining |
| | Schirmer's Test Safety Assessments Randomization |
| | In-Office Dose & Drop Comfort Questionnaire Study Drug Dispensed |
| Visit 2 | Collection of Study Drug Medical/Medication Update: AE Query |
| (Day 8 ± 1): | Visual Acuity |
| 1-Week Follow-Up | Slit Lamp Exam TFBUT, Fluorescein Staining |
| | Safety Assessments |
| | In-Office Dose & Drop Comfort Questionnaire Study Drug Dispensed |
| Visit 3 | Collection of Study Drug Medical/Medication Update: AE Query Tear |
| (Day 29 ± 2): | Symptom Questionnaires, Visual Acuity Slit Lamp Exam |
| 4-Week Follow-Up | Tear Osmolarity |
| and Study Exit | Tear Collection |
| | TFBUT, Fluorescein and Lissamine Staining, Schirmer's Test |
| | Safety Assessments, Study Exit |

Fifty-one subjects were enrolled, for a total of 17 subjects per trial arm. Subjects were randomized 1:1:1 to receive ADX-102 Ophthalmic Solution (0.1%), ADX-102 Ophthalmic Solution (0.5%), or ADX-102 Ophthalmic Lipid Solution (0.5%). A vehicle control was not included in the clinical trial.

Thirty-nine subjects completed the trial: 16 subjects in the ADX-102 Ophthalmic Solution (0.1%) group; 12 subjects in the ADX-102 Ophthalmic Solution (0.5%) group; and 11 subjects in the ADX-102 Ophthalmic Lipid Solution (0.5%) group. Twelve subjects did not complete the study (D/C subjects).

Subjects self-administered ADX-102 Ophthalmic Solution four times per day (morning, noon, afternoon, and before bed) throughout the study. Subjects did not use study drug prior to Study Visits.

MDA adduct ELISA. Normal human tears, pooled from three individuals (two males and one female), were purchased from Bioreclamation IVT (catalog number hmtears). The MDA adduct ELISA kit is commercially available and was purchased from Cell Biolabs, Inc., San Diego, Calif. (OxiSelect MDA Adduct Competitive ELISA, catalog number STA-832).

The assay is a competitive ELISA. An MDA conjugate is adsorbed onto an ELISA plate. Samples containing unknown amounts of MDA adducts or MDA-BSA standards are then added to the plate and incubated. An MDA antibody is then added to the plate, followed by an HRP-labelled secondary antibody. The plate is washed and an HRP detection agent is added. The plate is read in a microplate reader at 450 nm. The assay OD reading decreases with increasing MDA adducts in the samples, as the adsorbed MDA competes for binding to the MDA antibody with MDA adducts in the test sample.

A standard curve for the assay was generated using 0, 0.025, 0.05, 0.10, 0.20, 0.39, 0.78, 1.56, 3.13, and 6.25 μg/mL of MDA-BSA. Standard and unknown samples volumes in the assay were 50 μL each.

Pilot Assay. In addition to the standard curve, neat NHT samples and NHT samples spiked with MDA-BSA were measured. NHT spiked with 12,000 pmol/mL concentration of the internal standard, MDA-BSA, was used to determine dilutional integrity. D/C samples and NHT spiked samples were diluted 20- and 80-fold in PBS buffer containing 0.1% BSA prior to assay. Neat NHT samples were diluted 20-fold to serve as a baseline reference.

DES Study Samples. Based on the results of the pilot assay, a 1:60 dilution was determined to maximize the likelihood of the OD values falling within the linear portion of the standard curve, and thus provide the most accurate results. All tear samples from subjects (0.5% ADX-102 Ophthalmic Solution) and (0.1% ADX-102 Ophthalmic Solution) were analyzed, but results from some tear samples were excluded from the data analysis due to contamination with ocular staining dye in the Visit 3 samples. The Visit 3 tear sample from some subjects (0.5% ADX-102 Ophthalmic Solution) was excluded from the data analysis due to insufficient volume.

Results—Pilot assay of D/C subject and NHT. The detection range of the assay was 6 to 1500 nM, and the linear range of the assay was approximately 10 to 110 nM. Normal human tears diluted 20-fold had an average calculated MDA adduct concentration of 2,266 pmol/mL (2,266 nM). Approximately 100% of the spiked MDA-BSA adduct (12,000 pmol/mL) was recovered in NHT. Tears from D/C DES subjects had a mean MDA adduct concentration of 7,798 pmol/mL, a 3.4-fold increase relative to NHT; this difference was statistically significant (FIG. 1).

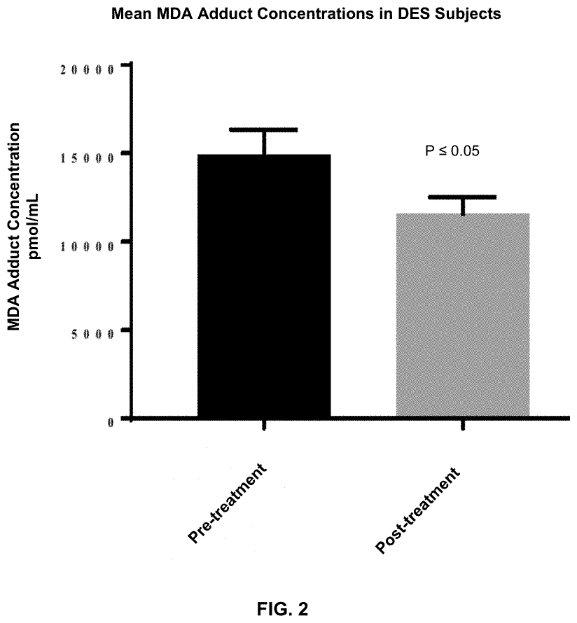

Assay of tears from subjects who completed the trial. FIG. 2 shows the calculated mean MDA adduct concentrations in tears from 37 DES subjects who completed the trial, at Visit 1 (baseline, before treatment) and Visit 3 (after 28 days of treatment with ADX-102 Ophthalmic Solution). Two subjects were excluded from this analysis because corneal fluorescein staining was conducted prior to tear collection, which interfered with the MDA adduct ELISA signal. Tears collected at Visit 1 had a mean MDA adduct concentration of 14,943 pmol/mL, which is significantly higher than the mean MDA adduct concentration of 11,566 pmol/mL in tears collected from all subjects at Visit 3.

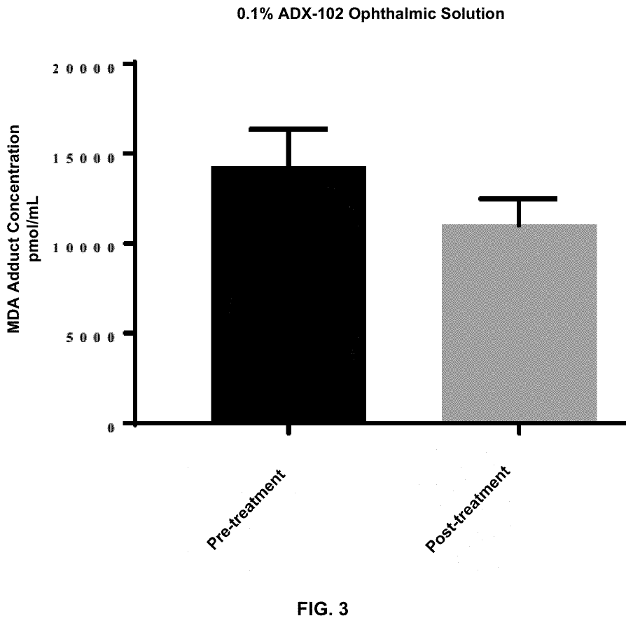

FIG. 3 shows MDA adduct concentrations in tears in subjects treated with 0.1% w/v ADX-102 Ophthalmic Solution Subjects treated with ADX-102 Ophthalmic Solution (0.1%) had a mean MDA adduct concentration of 14,287 pmol/mL at Visit 1, compared to 11,028 pmol/mL at Visit 3, which corresponds to a 23% reduction in MDA adduct levels after treatment.

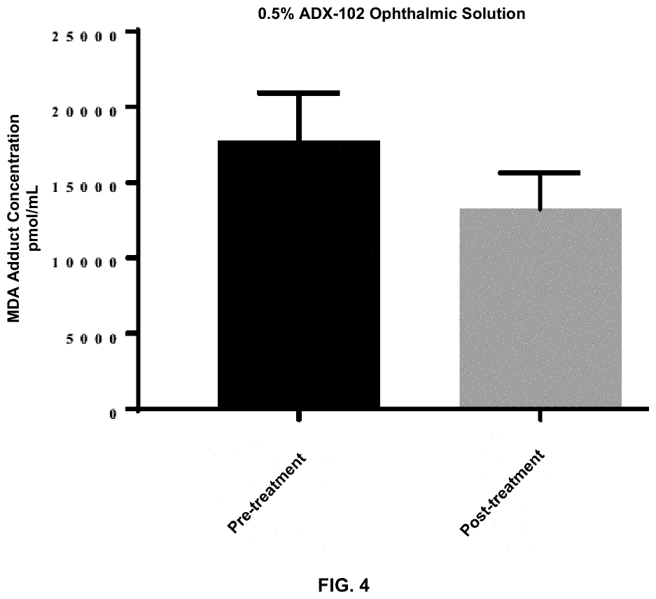

FIG. 4 shows MDA adduct concentrations in tears in subjects treated with ADX-102 Ophthalmic Solution (0.5%). Subjects treated with ADX-102 Ophthalmic Solution (0.5%) showed a 26% reduction in MDA adduct concentration at Visit 3 compared to Visit 1.

Although individually all treatment groups had lower MDA adduct concentrations on Visit 3 compared to Visit 1, the differences were not statistically significant.

Discussion MDA adducts were detected in tear samples collected from all DES subjects at Visit 1 and at Visit 3. MDA adduct concentrations were significantly lower on Visit 3 after a 4-week treatment with ADX-102 Ophthalmic Solution, relative to pre-treatment values on Visit 1, as shown in FIG. 1. Within each treatment group, MDA adduct levels in tears decreased on Visit 3 relative to Visit 1, but did not reach statistical significance. This finding may be related to the high variability in MDA adduct concentrations in DES subjects before treatment, combined with the small sample number. In addition, since the time between the last administration of study drug and collection of tears varied among subjects (estimated to be approximately eight to 12 hours), the timing of sample collection, relative to last treatment, may introduce variability into the post-treatment results at Visit 3.

The data show that MDA adduct levels are significantly higher in subjects with DES and are consistent with literature describing elevated MDA levels in tears of DES patients, compared to normal subjects. Furthermore, the data suggest that treatment with ADX-102 Ophthalmic Solution decreases MDA adducts in tears from subjects with DES.

### Example 3: A Phase 2/3, Multi-Center Randomized, Double-Masked, Parallel Design, Vehicle-Controlled Clinical Trial to Assess the Efficacy and Safety of 0.25% Reproxalap Ophthalmic Solution Compared to Vehicle in Subjects with Dry Eye Disease

| | |
|---|---|
| Protocol Title: | A Multi-Center Randomized, Double-Masked, Parallel |
| | Design, Vehicle-Controlled Phase 2/3 Clinical Trial to |
| | Assess the Efficacy and Safety of 0.25% Reproxalap |
| | Ophthalmic Solution Compared to Vehicle in Subjects with |
| | Dry Eye Disease |
| Investigational Product: | 0.25% Reproxalap Ophthalmic Solution (reproxalap) |
| Study Phase: | 2/3 |
| Primary Objective(s): | To evaluate the efficacy of reproxalap, as assessed by |
| | conjunctival redness, tear RASP levels, Schirmer's Test, |
| | and symptoms after dosing prior to and during exposure to |
| | the Controlled Adverse Environment ® (CAE) in subjects |
| | with dry eye disease |
| Overall Study Design: | |
| Structure: | Multi-center, double-masked, randomized parallel design |
| | trial |
| Duration: | An individual subject's participation is estimated to be |
| | approximately 16-32 days. |
| Controls: | Vehicle Ophthalmic Solution (vehicle) |
| Dosage/Dose Regimen: | Test article (reproxalap or vehicle) will be dosed topically |
| | in both eyes. |
| | Test article will be administered QID on Day 1 (Visit |
| | 2). On Day 2 (Visit 3), test article will be |
| | administered once within 10 minutes prior to the CAE ® |
| | entry, once 45 minutes after initiation of the CAE ®, and |
| | once at CAE ® exit. |
| Summary of Visit Schedule: | Three visits over the course of approximately 2 weeks: |
| | Visit 1 = Day −14 −16/+2, Screening |
| | Visit 2 = Day 1, Randomization/Baseline |
| | Visit 3 = Day 2 CAE ® & Study Exit |
| | Twenty subjects who meet the enrollment criteria will |
| | participate in an Initial Cohort. The Initial Cohort phase |
| | will be limited to Visit 1 (Screening), Visit 2 (Day 1), and |
| | Visit 3 (Day 2). Subjects will be randomized 1:1 to receive |
| | either reproxalap or vehicle. Results from the Initial |
| | Cohort phase will be analyzed to confirm endpoints, and |
| | statistical power for the remainder of the trial. |
| Measures Taken to Reduce | This trial is a randomized treatment assignment, double- |
| Bias: | masked trial. |
| Study Population | |
| Characteristics: | |
| Number of Subjects: | Twenty subjects are expected to be enrolled in the Initial |
| | Cohort of the trial. |
| | Approximately 300 subjects are expected to be enrolled in |
| | the Main Cohort of the trial. |
| Condition/Disease: | Dry Eye Disease (DED) |
| Inclusion Criteria: | Subjects must meet all of the following criteria: |
| | 1. 18 years of age (either gender and any race); |
| | 2. Ability to provide written informed consent and |
| | sign the Health Information Portability and Accountability |
| | Act (HIPAA) form; |
| | 3. Reported history of dry eye for at least 6 months |
| | prior to Visit 1; |
| | 4. Reported history of use or desire to use eye drops |
| | for dry eye symptoms within 6 months of Visit 1; |
| | 5. Corneal fluorescein staining sum (sum of inferior, |
| | superior, and central) in at least one eye on the Ora |
| | Calibra Scale at Visit 1. |
| | 6. Response to the CAE ® at Visit 1, as defined by: |
| | a. A ≥15-point increase in the visual analog scale eye |
| | dryness score (0-100) and a ≥0.5-point increase in |
| | conjunctival redness score in at least one eye during at |
| | least two consecutive Subjects must not meet any of the |
| | following criteria: |
| | 1. Clinically significant slit lamp findings at Visit 1 |
| | that may include active blepharitis, meibomian gland |
| | dysfunction (MGD), lid margin inflammation, or active |
| | ocular allergies that require therapeutic treatment, and/or |
| | in the opinion of the investigator may interfere with study |
| | parameters; |
| | 2. Diagnosis of an ongoing ocular infection |
| | (bacterial, viral, or fungal), or active ocular inflammation |
| | at Visit 1; |
| | 3. Contact lens use within 7 days of Visit 1 or |
| | anticipate using contact lenses during the trial; |
| | 4. Eye drop use within 2 hours of Visit 1; |
| | 5. Previous laser-assisted in situ keratomileusis |
| | (LASIK) surgery within the last 12 months; |
| | 6. Cyclosporine 0.05% or 0.09% or lifitegrast 5.0% |
| | ophthalmic solution use within 90 days of Visit 1; |
| | 7. Systemic corticosteroid or other |
| | immunomodulator therapy (not including inhaled |
| | corticosteroids) within 14 days of Visit 1 or any planned |
| | immunomodulatory therapy throughout the study period; |
| | 8. Planned ocular and/or lid surgeries over the study |
| | period or any ocular surgery within 6 months of Visit 1; |
| | 9. Temporary punctal plugs during the study that |
| | have not been stable within 30 days of Visit 1; |
| | Use of and unwillingness to discontinue topical |
| | ophthalmic prescription (including medications for |
| | glaucoma) or over-the-counter (OTC) solutions, artificial |
| | tears, gels, or scrubs for the duration of the trial (excluding |
| | medications allowed for the conduct of the trial); |
| | 11. Corrected visual acuity greater than or equal to |
| | logarithm of the minimum angle of resolution (logMAR) + |
| | 0.7 as assessed by Early Treatment of Diabetic |
| | Retinopathy Study (ETDRS) scale in both eyes at Visit 1; |
| | 12. Pregnancy, nursing, or planned pregnancy during |
| | the conduct of the trial; |
| | 13. Unwillingness io submit a urine pregnancy test at |
| | Visit 1 and Visit 3 (or early termination visit) if of |
| | childbearing potential. (Non-childbearing potential is |
| | defined as a woman who is permanently sterilized [e.g., |
| | has had a hysterectomy or tubal ligation], or is post- |
| | menopausal [without menses for 12 consecutive months]); |
| | 14. If of childbearing potential (female or male), |
| | unwillingness io use an acceptable means of birth control. |
| | (Acceptable methods of contraception include: hormonal - |
| | oral, implantable, injectable, or transdermal |
| | contraceptives; mechanical - spermicide in conjunction |
| | with a barrier such as a diaphragm or condom; intrauterine |
| | device [IUD]; or surgical sterilization of partner. For non- |
| | sexually active males or females, abstinence may be |
| | regarded as an adequate method of birth control; however, |
| | if the subject becomes sexually active during the study, |
| | he/she must agree io use adequate birth control as defined |
| | above for the remainder of the trial.); |
| | 15. Known allergy and/or sensitivity to the test article |
| | or its components; |
| | 16. A condition that the investigator feels may put the |
| | subject at significant risk, may confound the study results, |
| | or may interfere significantly with the subject's |
| | participation in the trial; |
| | Current enrollment in an investigational drug or device |
| | study or have used an investigational drug or device |
| | within 30 days of Visit 1; |
| | 18. Use of reproxalap ophthalmic solution in the past |
| | year; |
| | 19. Current use of any medication known to cause |
| | ocular drying that is not used on a stable dosing regimen |
| | for at least 30 days prior to Visit 1; |
| | 20. Inability or unwillingness to follow instructions, |
| | including participation in all study assessments and visits. |
| Evaluation Criteria: | |
| Primary Endpoint | Conjunctival redness assessed via digital photography |
| | over 90 minutes in CAE |
| Secondary Endpoints: | Visual analog scale eye dryness score assessed over 24 |
| | hours after first dose on Day 1, and over 90 minutes |
| | in CAE ® |
| | Ora Calibra ® Ocular Discomfort Scale assessed over |
| | 24 hours after first dose on Day 1, and over 90 |
| | minutes in CAE ® |
| | Ocular Discomfort & 4-Symptom Questionnaire |
| | assessed over 24 hours after first dose on Day 1, and |
| | before and after CAE |
| | Ora Calibra ® Conjunctival Allergen Challenge Ocular |
| | Itching Scale assessed over 24 hours after first dose |
| | on Day 1, and before and after CAE |
| | Schirmer's Test change from baseline before and after |
| | the final dose on Day 1 |
| | Change in tear RASP levels before and after Dose 1 |
| | and 2 on Day 1 and Dose 3 on Day 2 |
| | Conjunctival Redness over 24 hours after first dose on |
| | Day 1 |
| Safety Endpoints: | Visual acuity |
| | Slit-lamp evaluation |
| | Adverse event query |
| | Intraocular Pressure (IOP) |
| | Dilated fundoscopy |

General Statistical Methods and Types of Analyses:

Changes over 24 hours after the first dose on Day 1 and changes over 90 minutes in the CAE will be assessed via mixed effect model for repeated measures (MMRM) of change from baseline (pre-dose Day 1), with baseline score as a covariate, and time and test article group as factors. For assessments that include both eyes, eye will be added as a factor.

Schirmer's Test will be assessed via MMRM of change from baseline (screening), with baseline score as a covariate, and pre/post dose, eye, and test article group as factors.

Tear RASP levels will be assessed via MMRM of change from pre to post dose on Day 1 and Day 2, with baseline (pre-dose) scores as a covariate, and dose and test article group as factors.

Safety endpoints will be summarized using descriptive statistics.

Statistical analyses will be detailed in the Statistical Analysis Plan (SAP), which will dominate any statistical language herein.

### Abbreviations

AE adverse event
CAE Controlled Adverse Environment®
CFR Code of Federal Regulations
DED dry eye disease
DES dry eye syndrome
DHHS Department of Health and Human Services
ETDRS Early Treatment of Diabetic Retinopathy Study
FDA Food and Drug Administration
GCP Good Clinical Practice
HIPAA Health Information Portability and Accountability Act
ICF informed consent form
ICH International Council for Harmonisation
IEC Independent Ethics Committee
IRB institutional/independent review board
Log MAR logarithm of the minimum angle of resolution
Ora Ophthalmic Research Associates, Inc.
OTC over the counter
RASP reactive aldehyde species
SAE serious adverse event
μL microliter
VA visual acuity
VAS Visual analog scale

The clinical objectives for this Phase 2/3 study are to evaluate the efficacy of reproxalap, as assessed by conjunctival redness, tear RASP levels, Schirmer's Test, and symptoms after dosing prior to and during exposure to the Controlled Adverse Environment® (CAE) in subjects with dry eye disease.

Overall Study Design

This study is a Phase 2/3, multi-center, randomized, double-masked, parallel design, vehicle-controlled trial designed to evaluate the efficacy and safety of 0.25% Reproxalap Ophthalmic Solution compared to vehicle in subjects with dry eye disease. Approximately twenty subjects will be enrolled in the Initial Cohort, and approximately 300 subjects will be enrolled in the Main Cohort. Male and female subjects at least 18 years of age with a subject-reported history of dry eye disease in both eyes and meeting all other eligibility criteria will be randomized to receive reproxalap or vehicle in a 1:1 ratio (approximately 150 subjects in each treatment group).

This study uses a challenge-model of dry eye disease known as a “dry eye chamber.” Challenge-model trials utilizing a controlled chamber are an FDA accepted design for pivotal endpoints. See, for example, FDA published “Dry Eye: Developing Drugs for Treatment” draft guidance; December 2020.

Dry eye chambers control relative humidity, temperature, airflow, and visual tasking in order to stress the ocular surface. Chambers simulate a “bad day” scenario in the life of a dry eye disease sufferer. Trial designs utilizing chambers are able to confirm the utility of drugs with rapid onset of action during an acute ocular surface challenge.

Order of Events:

| | |
|---|---|
| Visit 1 | Informed Consent |
| (Day −14 −16/+2): | Demographics, Medical/Medication & |
| Screening | Ocular History |
| | Urine Pregnancy Testing (as needed) |
| | Symptom Questionnaires, Ocular Dryness VAS, |
| | Visual Acuity |
| | Ora Calibra ® Conjunctival Allergen Challenge |
| | Ocular Itching Scale |
| | SlitLamp Exam with Conjunctival Redness |
| | assessment |
| | In-Office vehicle dose (within 10 minutes prior |
| | to CAE entry) |
| | Ora Calibra ® Corneal and Conjunctival Staining |
| | Scale CAE exposure (90 minutes) with Ocular |
| | Dryness VAS and Ora Calibra ® Ocular Discomfort |
| | Scale |
| | SlitLamp Exam with Conjunctival Redness |
| | assessment |
| | Schirmer's Test |
| | IOP and Dilated Fundoscopy |
| Visit 2 (Day 1): | Medical/Medication Update: AE Query |
| Randomization/ | Visual Acuity |
| Baseline | Slit Lamp Exam |
| | Symptom Questionnaires at specified time |
| | points |
| | Conjunctival Redness at specified time points |
| | In-Office Doses (QID) |
| | Tear Collection & Schirmer's Test at specified |
| | time points |
| Visit 3 (Day 2): | Medical/Medication Update: AE Query |
| CAE | Urine Pregnancy Testing (as needed) |
| | Visual Acuity, Slit Lamp Exam |
| | Symptom Questionnaires |
| | Conjunctival Redness at specified time points |
| | In-Office Doses (within 10 minutes prior to CAE |
| | entry, 45 minutes after CAE entry, and at CAE exit) |
| | CAE exposure (90 minutes) with Ocular Dryness |
| | VAS, and Ora Calibra ® Ocular Discomfort Scale, |
| | and Conjunctival Redness Assessments |
| | Tear Collection at specified time points |
| | IOP and Dilated Fundoscopy |
| | Study Exit |

Study Parameters

Primary Endpoint: Conjunctival redness assessed via digital photography over 90 minutes in CAE®

Safety Endpoints:
Visual acuity
Slit-lamp evaluation
Adverse event query
Intraocular Pressure (IOP)
Dilated fundoscopy

Secondary Endpoints:
Visual analog scale eye dryness score assessed over 24 hours after first dose on Day 1, and over 90 minutes in CAE® (Key Secondary Symptom Endpoint)
Ora Calibra® Ocular Discomfort Scale assessed over 24 hours after first dose on Day 1, and over 90 minutes in CAE®
Ocular Discomfort & 4-Symptom Questionnaire assessed over 24 hours after first dose on Day 1, and before and after CAE®
Ora Calibra® Conjunctival Allergen Challenge Ocular Itching Scale assessed over 24 hours after first dose on Day 1, and before and after CAE®
Schirmer's Test change from baseline before and after the final dose on Day 1
Change in tear RASP levels before and after Dose 1 and 2 on Day 1 and Dose 3 on Day 2. Conjunctival redness over 24 hours after first dose on Day 1.

Regarding the use of tear RASP levels as a clinical endpoint, it is important to note that this is (to date) unprecedented. The FDA has not agreed to the use of RASP before as a clinical endpoint. In this regard, we have reached agreement with the US Food and Drug Administration (FDA) for the use of RASP (reactive aldehyde species) as an objective sign for the treatment of dry eye disease. RASP are pre-cytokine pro-inflammatory mediators that are elevated in the tears of patients with dry eye disease, and correlate with dry eye disease symptoms and signs. In a Phase 2a dry eye disease clinical trial, reproxalap demonstrated reduction in tear RASP levels following 28 days of treatment. In in vitro studies, RASP were eliminated within 60 to 90 minutes when exposed to reproxalap at equimolar concentrations. Reproxalap, when administered topically to the eye, is thought to be more than 500-fold in excess of tear RASP levels, and has demonstrated consistent statistically significant and clinically relevant activity in dry eye disease, allergic conjunctivitis, and other forms of ocular inflammation across numerous Phase 2 and Phase 3 clinical trials. The use of RASP as a trial endpoint represents the first novel objective sign for the treatment in dry eye disease in over a decade.

Tear RASP levels from the TRANQUILITY run-in cohort were reduced after single doses of the novel RASP inhibitor reproxalap, as assessed by enzyme-linked immunosorbent assay (ELISA) of 4-hydroxynonenal protein adducts (HNE), a RASP selected based on results from a natural history study of dry eye patients conducted by Aldeyra. For subjects with sufficient tear volumes for analysis, across the two doses where tear RASP levels were assessed before and after drug administration, HNE levels declined by an average of 1018 picograms/milliliter (pg/mL) in reproxalap-treated patients (n=9) versus an increase of 32 pg/mL in vehicle-treated patients (n=7). Accordingly, tear RASP levels have been selected as a secondary endpoint for confirmation of mechanism of action. HNE is well-characterized in the scientific literature as a critical pro-inflammatory RASP,¹ and ocular levels of HNE correlate with the signs and symptoms of dry eye disease.

Detailed Study Procedures

Examination Procedures

Procedures to be Performed at the Study Visit with Regard to Study Objective(s)

Visit 1 (Day −14 −16/+2, Screening)
Informed Consent and HIPAA
Demographics (e.g. gender, date of birth, race, ethnicity)
Medical/Medication & Ocular History
Urine Pregnancy Test (as needed)
Symptom Questionnaires:
   a. Ocular Discomfort & 4-Symptom Questionnaire
   b. Ora Calibra® Ocular Discomfort Scale
   c. Ocular Dryness Visual Analog Scale
   d. Ora Calibra® Conjunctival Allergen Challenge Ocular Itching Scale
Visual Acuity (ETDRS)
Slit Lamp Biomicroscopy with Conjunctival Redness assessment
Fluorescein Staining using the Ora Calibra® Corneal and Conjunctival Staining Scale
Inclusion/Exclusion Criteria Review
In-Office Vehicle Instillation by trained site staff (within 10 minutes prior to CAE® entry)
CAE® Exposure (90 minutes, vehicle administration at 45 minutes) with:
   a. Ocular Dryness Visual Analog Scale every 5 minutes
   b. Ora Calibra® Ocular Discomfort Scale every 5 minutes
   c. Conjunctival Redness Photography at 0, 5, 10, 15, 20, 30, 45 (before 45-minute dose), 50, 55, 60, 65, 75, 90 minutes (+/−2 minutes) via digital photography
Slit Lamp Biomicroscopy with Conjunctival Redness assessment
Schirmer Test
Intraocular Pressure
Dilated Exam
Schedule for Visit 2

Visit 2 (Day 1, Baseline and In-Office Dosing)
Medical/Medication Update
Adverse Event Query
Visual Acuity (ETDRS)
Slit Lamp Biomicroscopy
Symptom Questionnaires
   a. Ocular Discomfort & 4-Symptom Questionnaire
   b. Ora Calibra® Ocular Discomfort Scale
   c. Ocular Dryness Visual Analog Scale
   d. Ora Calibra® Conjunctival Allergen Challenge Ocular Itching Scale
Conjunctival Redness Photography
Inclusion/Exclusion Criteria Review
Randomization/Enrollment
Pre-dose #1 Tear Collection (30+/−5 minutes pre-dose)
In-Office Dose #1
Post-dose #1 Tear Collection (started within 5+/−2 minutes of dose)
   Wait 3 hours between doses
Pre-dose #2 Tear Collection (30+/−5 minutes pre-dose)
In-Office Dose #2
Post-dose #2 Tear Collection (started within 5+/−2 minutes of dose)
   Wait 30 minutes between doses
In-Office Dose #3
Conjunctival Redness Photography (10 minutes+/−2 minutes post-dose #3)
Symptom Questionnaires started within 10 minutes+/−2 minutes Post-dose #3:
Ocular Discomfort & 4-Symptom Questionnaire
Ocular Discomfort
Ocular Dryness Visual Analog Scale
Ora Calibra® Conjunctival Allergen Challenge Ocular Itching Scale
   Wait 30 minutes between doses
Pre-dose #4 Schirmer's Test (10+/−2 minutes pre-dose)
In-Office Dose #4
Post-dose #4 Schirmer's Test (started within 5+/−2 minutes of dose)
Schedule for Visit 3
Visit 3 (Day 2, CAE®)
Medical/Medication Update
Adverse Event Query
Urine Pregnancy Test (as needed)
Visual Acuity (ETDRS)
Slit Lamp Biomicroscopy
Conjunctival Redness Photography
Symptom Questionnaires:
   a. Ocular Discomfort & 4-Symptom Questionnaire
   b. Ora Calibra® Ocular Discomfort Scale
   c. Ocular Dryness Visual Analog Scale
   d. Ora Calibra® Conjunctival Allergen Challenge Ocular Itching Scale
Pre-dose #1 Tear Collection (30+/−5 minutes pre-dose)
In-Office Dose #1 within 10 minutes prior to CAE Entry
CAE Exposure (90 minutes) with:
Ocular Dryness Visual Analog Scale every 5 minutes
Conjunctival Redness at 0, 5, 10, 15, 20, 30, 45 (before 45-minute dose), 50, 55, 60, 65, 75, 90 minutes (+/−2 minutes) via digital photography
Ora Calibra® Ocular Discomfort Scale every 5 minutes
In-Office Dose #2 (45+/−2 minutes into CAE exposure, administer by site staff)
Post-CAE exit Tear Collection (started within 5+/−2 minutes)
In-Office Dose #3 (immediately following tear collection)
Post-dose #3 Tear Collection (started within 5+/−2 minutes of dose)
Symptom Questionnaires started within 10 minutes+/−2 minutes post dose #3:
   a. Ocular Discomfort & 4-Symptom Questionnaire
   b. Ocular Discomfort
   c. Ocular Dryness Visual Analog Scale
   d. Ora Calibra® Conjunctival Allergen Challenge Ocular Itching Scale
Slit Lamp Biomicroscopy
Intraocular Pressure
Dilated Exam
Study Exit

Statistical Hypotheses

The following hypothesis will be tested comparing reproxalap to vehicle. The null hypotheses must be rejected for the dosing regimen to claim efficacy.

H01: There is no difference between reproxalap and vehicle in the overall mean change from baseline in conjunctival redness.

H11: The overall mean improvement from baseline in conjunctival redness levels is larger with reproxalap than with vehicle.

Sample Size

Based on the conjunctival redness results from the Initial Cohort, 150 subjects per arm are required for 90% power to detect a statistically significant difference between arms, assuming a difference between groups of approximately 0.1 units and a standard deviation of the difference of 0.3 units.

Statistical Analysis

General Considerations:

Quantitative variables will be summarized descriptively using number of subjects (n), mean, standard deviation, median, minimum, and maximum. Qualitative variables will be summarized using counts and percentages.

All summaries will be presented by treatment group Summaries will be provided for demographics, baseline medical history, concurrent therapies, and subject disposition.

For the purpose of summarization, medical history, concurrent therapies, and adverse events will be coded to MedDRA and WHODrug dictionaries, as appropriate.

Baseline measures are defined as the last non-missing measure prior to the initiation of randomized study treatment at Day 1. Change from baseline will be calculated as follow-up visit value minus baseline value. Treatment comparisons between active and vehicle will be matched by dosing regimen and calculated as active minus vehicle.

All analyses will be 2-sided at a significance level of 0.05. 95% confidence intervals will be provided where appropriate.

The statistical analysis plan (SAP) will detail the statistical procedures, and will dominate any text herein.

Unit of Analysis:

Safety endpoints will be analyzed for both eyes. RASP assessments will be made on tear samples pooled across both eyes; redness, Schirmer's Test, and Ocular Discomfort Scores will be collected and analyzed for each eye; and VAS dryness and Ocular Discomfort & 4-Symptom Questionnaire scores will be collected for both eyes in aggregate. Assessment scales are detailed in the Appendices.

Missing Data:

As sensitivity measures, efficacy analyses may be conducted with multiple imputation under missing at random (MAR) and missing not at random (MNAR) assumptions. Per-protocol population analysis may also be conducted to assess sensitivity. Further detail will be described in the SAP.

Multiplicity Considerations:

No multiplicity corrections will be required.

Primary Efficacy Analysis:

The primary efficacy analysis of change from baseline in conjunctival redness over all time points in the CAE will be analyzed via MMRM, with baseline score as a covariate, and eye, time, and test article group as factors.

Secondary Efficacy Analyses:

Changes over 24 hours after the first dose on Day 1 and changes over 90 minutes in the CAE will be assessed via mixed effect model for repeated measures (MMRM) of change from baseline (pre-dose Day 1), with baseline score as a covariate, and time and test article group as factors. For assessments that include both eyes, eye will be added as a factor. Schirmer's Test will be assessed via MMRM of change from baseline (screening), with baseline score as a covariate, and pre/post dose, eye, and test article group as factors. Tear RASP levels will be assessed via MMRM of change from pre to post dose on Day 1 and Day 2, with baseline (pre-dose) scores as a covariate, and dose and test article group as factors.

Results

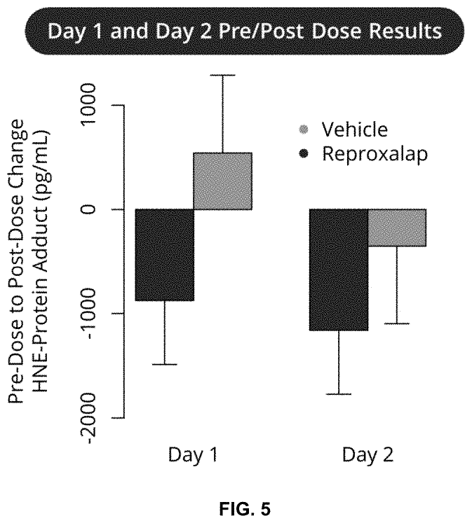

Data for the run-in cohort was obtained and analyzed. FIG. 5 shows reduction in HNE-protein adduct levels in DED patient tears. The data show Day 1 and Day 2 pre/post dose results (pre-dose to post-dose change) in HNE-protein adduct levels (pg/mL) in the patients dosed with either vehicle or reproxalap. Day 1 dose is first dose of Day 1. Day 2 dose is dose post chamber. Tear collections taken approximately 10 minutes before and after dosing.

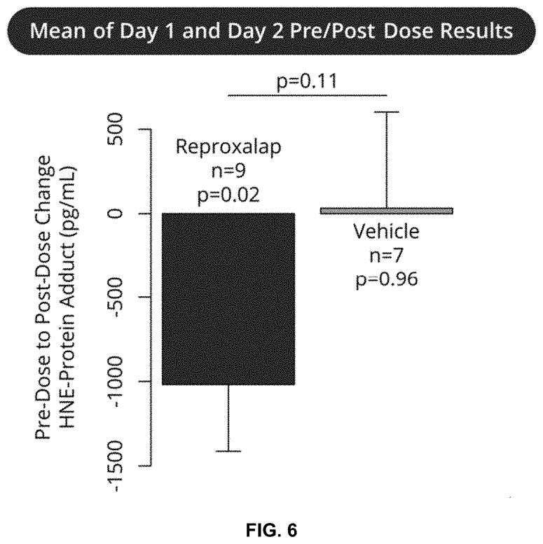

FIG. 6 shows the mean of reduction in HNE-protein adduct levels in DED patient tears. P values by group represent difference from 0 (no change). Means represent average of the two doses where tear RASP were assessed before and after dosing. HNE=4-hydroxynonenal ELISA of protein adducts. Tear RASP levels from the Phase 3 clinical trial run-in cohort were reduced after single doses of the novel RASP inhibitor reproxalap, as assessed by enzyme-linked immunosorbent assay (ELISA) of 4-hydroxynonenal protein adducts (HNE), a RASP selected based on results from a natural history study of dry eye patients. For subjects with sufficient tear volumes for analysis, across the two doses where tear RASP levels were assessed before and after drug administration, HNE levels declined by an average of 1018 picograms/milliliter (pg/mL) in reproxalap-treated patients (n=9) versus an increase of 32 pg/mL in vehicle-treated patients (n=7).

Regarding symptoms of DED, statistical significance was achieved for both sign and symptoms in dry eye chamber, as shown in Table 3 below:

TABLE 3

| | Change From Baseline | | |
|---|---|---|---|
| Dry Eye Assessment (Scale) | Reproxalap | Vehicle | |
| After Environmental Dosing | (N = 12) | (N = 11) | P-Value |
| VAS Dryness (0-100) | −26 | +2 | 0.003 |
| OD4S: Discomfort (0-5) | −0.7 | +0.4 | 0.003 |
| OD4S: Dryness (0-5) | −1.2 | +0.1 | 0.006 |
| OD4S: Grittiness (0-5) | −1.1 | +0.1 | 0.006 |
| OD4S: Burn (0-5) | −0.1 | +0.8 | 0.07 |
| OD4S: Sting (0-5) | −0.1 | +0.4 | 0.23 |
| Ocular Discomfort Scale (0-4) | −0.7 | +0.4 | 0.07 |
| Schirmer's Test (mm)* | +3.4 | +1.3 | 0.30 |

*Day 1 Schirmer's Test results based on improvement after a single dose; all other Day 1 assessments performed over 24 hours of QID dosing. Topical ocular reproxalap has been studied in over 1,100 patients thus far with no observed safety concerns; mild instillation site irritation is the most commonly reported adverse event in clinical trials.
Source: TRANQUILITY Run-In Cohort initial results. VAS = Visual Analog Scale; OD4S = Ocular Discomfort & 4-Symptom Questionnaire; QID = Four times daily.

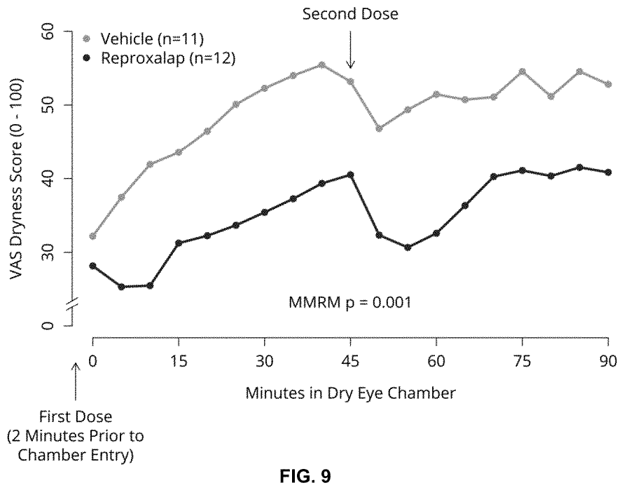

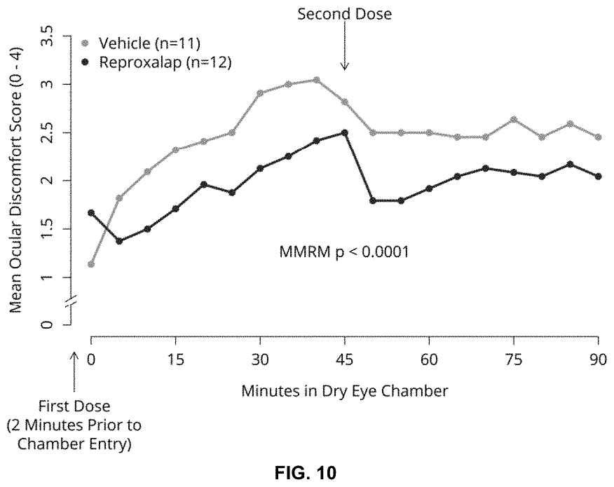

Notable Results:
Reproxalap demonstrated statistically significant improvement over vehicle in ocular redness (p=0.03), an FDA-approvable objective sign.
Reproxalap demonstrated statistically significant improvement over vehicle for the two assessed clinical symptoms: VAS Ocular Dryness (p=0.001) and Ocular Discomfort Score (p<0.0001).
Acute improvements in ocular symptoms and objective sign within minutes of reproxalap administration in a dry eye chamber.
24-hour environmental results supportive of reproxalap's rapid and broad efficacy, with consistent directional improvements over vehicle across symptoms and Schirmer's test, and statistical significance vs. vehicle achieved in four of eight assessed outcome measurements.
As shown in the TRANQUILITY run-in cohort Day 2 results (FIG. 9), drug effect over vehicle was observed upon entry and sustained throughout duration of exposure.
Therapeutic activity was demonstrated after first and second doses, representing near-immediate (within minutes) and statistically significant symptom relief vs. vehicle.
Consistent and statistically significant improvements over vehicle observed across all symptoms evaluated in the chamber.
As shown in FIG. 10, statistically significant drug effect over vehicle in ocular discomfort symptoms was observed upon entry and sustained throughout the duration of exposure. Again, therapeutic activity was demonstrated after first and second doses, representing near-immediate (within minutes) and statistically significant symptom relief

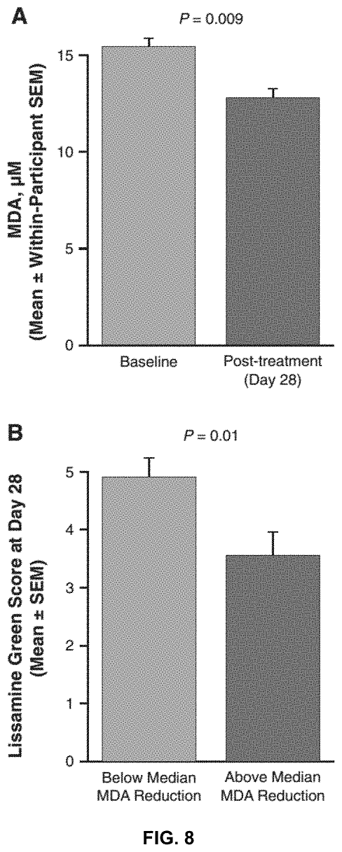

In addition, we have observed in previous Phase 2a studies that tear levels of MDA adduct were statistically lower after treatment (FIG. 8A). Participants with above-median reduction in MDA demonstrated statistically lower lissamine green staining scores than did participants with below-median reduction in MDA (FIG. 8B). Participants with above-median reduction in MDA adduct levels demonstrated statistically different tear osmolarity scores versus 0, whereas participants with below median MDA adduct reduction were not statistically different from 0. Supportive of the relationship of MDA adducts to osmolarity, reduction in osmolarity was correlated with reduction in MDA adduct levels (Pearson r=0.31, P=0.07). Results from the pooled reproxalap groups indicated that levels of MDA, a RASP previously described to be elevated in the tears of patients with DED, were statistically lower after 28 days of therapy than at baseline. Consistent with the clinical relevance of RASP as a proinflammatory mediator, reduction in MDA levels correlated with improvements in tear osmolarity and lissamine green staining RASP are upstream pre-cytokine potentiators of the innate immune response, including activation of NF-kB, inflammasomes, and scavenger receptor A, which may broadly exacerbate anterior segment inflammatory disease. Thus, RASP inhibition could explain the multifaceted activity of reproxalap observed across several signs and symptoms of DED. To our knowledge, the MDA findings represent the first direct clinical measurement of drug mechanism of action for any DED drug.

### Example 4: Additional Assays for RASP Levels in Biological Samples

We developed assays as described below to determine levels of RASP in biological samples such as patient tears.

### Summary

Notable Results:
Aldehydes (4-HNE, Pentanal, Hexanal, and Decanal) are amenable to trapping using semicarbazide.
Aldehyde conjugates of ADX-102 are present in treated clinical tears, ADX-102 API, and drug product. These conjugates appear to be at levels higher than endogenous aldehydes in human tears.
Use of semicarbazide will “scavenge” aldehyde from ADX-102 conjugates.
LC-MS analysis of ADX-102-4HNE conjugate has an LOQ of 5 nM (1.8 ng/mL).
Unknown aldehyde conjugates (C3, C4, C5, C6, C7, C8, C9, and C10) are detected in treated clinical tears, ADX-102 API, and drug product.

### Introduction

This report describes exploratory experiments conducted to evaluate aldehyde trapping as an analytical approach to determining free aldehyde levels in human tears. Several aldehydes and trapping agents, including reproxalap, were utilized in these studies.

### Results

Exploratory Trapping Studies in PBS and ACN/H₂O

Semicarbazide (SCZ), glutathione (GSH), cysteine, and N-acetyl cysteine were used as trapping agents with the aldehydes 4-hydroxynonenal (4-HNE), malondialdehyde, acetaldehyde, hexanal, pentanal, and decanal. 10 μM of aldehyde was mixed with 1 mM of trapping reagent in either PBS or 50/50 ACN/H₂O. As judged by MS, only semicarbazide and glutathione appeared to trap aldehydes (Decanal, Pentanal, Hexanal, and 4-HNE). SCZ was more effective than GSH.

Exploratory Trapping Studies in Tears

Semicarbazide (SCZ) and glutathione (GSH) were used as trapping agents with the aldehydes 4-hydroxynonenal (4-HNE), malondialdehyde, acetaldehyde, hexanal, pentanal, decanal, and acrolein. 50 μL of tears was spiked with trapping agent (final concentration 1 mM), incubated for 1 hour, and ACN was added (final percentage of ACN was 25%). Three aldehydes were detected by MS (hexanal, pentanal, and decanal).

Exploratory Trapping Studies in Tears Repeat

Semicarbazide (SCZ) was used as trapping agent with the aldehydes 4-hydroxynonenal (4-HNE), malondialdehyde, acetaldehyde, hexanal, pentanal, decanal, and acrolein. 50 μL of tears was spiked with trapping agent (final concentration 1 mM), incubated for 1 hour, and ACN was added (final percentage of ACN was 25%). Three aldehydes were detected using a longer gradient (hexanal, pentanal, and decanal). A putative endogenous butanal related aldehyde was also detected in the study at m/z=144.0773.

Aldehyde Stability Studies in Tears

The aldehydes hexanal, pentanal, and decanal were spiked into tears and trapped with d6-reproxalap (which is fully deuterated on both methyl groups of reproxalap) to test for trapping stability. A comparison with vehicle control samples indicated that hexanal, decanal, and pentanal were observed in the vehicle control samples at higher levels than in the samples where these aldehydes were spiked.

Aldehyde Presence in Plastic and Glass

A comparison of glass vs. plastic (as the source of aldehydes) was made using d₆-reproxalap solution. No apparent differences in aldehyde levels were detected.

Aldehyde Presence in Solvents

Water, ACN, and PBS were “cleaned” of aldehydes by addition of 0.5 mM semicarbazide (SCZ) and allowing 1 hour to react. 0.7 mM acetaldehyde was added to quench any remaining SCZ and allowed to react for 15 minutes.

A new solution of d₆-reproxalap (10 mM) was prepared using “clean” 1:1 ACN:Water. Aldehyde (Pentanal, Hexanal, and Decanal) conjugates of d₆-reproxalap were monitored in glass HPLC vials, with the use of no lid, foil, or plastic lids. The results were nearly identical for each, with pentanal, hexanal, and decanal detectable at similar levels.

Aldehyde (Pentanal, Hexanal, and Decanal) conjugates of d₆-reproxalap were monitored in “clean” PBS and water. “Clean” PBS and water without addition of d₆-reproxalap were also monitored. d₆-reproxalap showed detectable hexanal and decanal peaks down to a 50 uM concentration of d₆-reproxalap by MS. Reproxalap showed a detectable peak for pentanal and decanal at 1000 uM and a detectable peak for decanal down to 100 uM.

Aldehyde Presence in API and Drug Product

Serial dilution of d₆-reproxalap and reproxalap using “clean” water was also performed. Serial dilution of Drug Product using “clean” water/vehicle showed detectable amounts of pentanal and decanal at 2- and 20-fold dilution and decanal only at 200-fold dilution. Serial dilution of Drug Product using “clean” water showed detectable amounts of pentanal and decanal at 10-fold dilution and decanal only at 100-fold dilution.

Aldehyde Presence in Vehicle, Tears

Detecting semicarbazide covalent conjugates of aldehydes (Pentanal, Hexanal, 4-HNE, and Decanal) was performed in a sample of the vehicle and showed detectable levels of each. Incubation of semicarbazide with commercial tears also yielded detectable levels of all four conjugates.

Aldehyde Scavenging Using SCZ and d₆-Reproxalap

Formation of a conjugate with 4-HNE was tested using ADX-102, d₆-reproxalap and SCZ. Addition of SCZ to ADX-102-4-HNE conjugate results in conversion of ADX-102-4-HNE conjugate to SCZ-4-HNE conjugate. Addition of d₆-reproxalap to ADX-102-4-HNE conjugate results in conversion of ADX-102-4-HNE conjugate to d₆-reproxalap-4-HNE conjugate.

Addition of ADX-102 and d₆-reproxalap to vehicle did not afford additional aldehyde conjugates. Addition of SCZ to emotional tears generated SCZ aldehyde conjugates.

Presence of SCZ Aldehyde Conjugates in PBS

Addition of SCZ to PBS did not initially afford aldehyde conjugates. After 5 days SCZ Hexanal and SCZ Pentanal were detected. Using 50% ACN in extraction of the 5-day sample afforded similar results.

Presence of SCZ Aldehyde Conjugates in Tears

Addition of SCZ to untreated Clinical Tears did not initially afford aldehyde conjugates. After 5 days SCZ Hexanal and SCZ Pentanal were detected. Addition of SCZ to treated Clinical Tears did not initially afford aldehyde conjugates. After 5 days, SCZ Hexanal and SCZ Pentanal were detected.

Presence of ADX-102 Aldehyde Conjugates in PBS

Mining for the presence of ADX-102 aldehyde conjugates in PBS did not afford aldehyde conjugates. Using 50% ACN in extraction of the sample afforded similar results.

Presence of ADX-102 Aldehyde Conjugates in Tears

Mining for the presence of ADX-102 aldehyde conjugates in untreated clinical tears did not afford aldehyde conjugates. Using 50% ACN in extraction of the sample afforded similar results. Mining for the presence of ADX-102 aldehyde conjugates in treated clinical tears afforded ADX-102-Hexanal and ADX-102-Pentanal aldehyde conjugates. Using 50% ACN in extraction of the sample afforded similar results. Leaving these samples in the autosampler for 5 days resulted in elevated levels of ADX-102 aldehyde conjugates.

Aldehyde Scavenging of Drug Product Using SCZ

ADX-102-Hexanal and ADX-102-Pentanal levels did not appear to alter immediately after addition of SCZ, 2 hours after addition of SCZ or incubating overnight. Similar results were obtained using 50% ACN in the sample extraction.

Determining LOQ for SCZ and ADX-102 4HNE

Serial dilution of ADX-102-4HNE conjugate were made to determine the limit of quantitation to be 0.1 μM. Serial dilution of SCZ-4HNE conjugate were made to determine the limit of quantitation to be between 0.1 μM and 1 μM. Serial dilution of ADX-102-4HNE conjugate in 1:1 water:ACN afforded an LOQ of 5 nM or 1.8 ng/mL.

Detection of Unknown Aldehyde Conjugates

Several aldehyde conjugates of unknown structure were detected in treated clinical tears. A table of these aldehyde conjugates is presented below.

| m/z Found | Formula | Potential Aldehyde |
|---|---|---|
| 277.1121 | C15H17ClN2O | C3 |
| 291.1274 | C16H19ClN2O | C4 |
| 305.1432 | C17H21ClN2O | C5 |
| 319.1576 | C18H23ClN2O | C6 |
| 333.1734 | C19H25ClN2O | C7 |
| 333.175 | C19H25ClN2O | C8 |
| 347.1895 | C20H27ClN2O | C8-2H |
| 359.1895 | C21H27ClN2O | C9-2H |
| 359.194 | C21H27ClN2O | C9-2H |
| 361.2043 | C21H29ClN2O | C9 |
| 371.1891 | C22H27ClN2O | C10-4H |
| 373.2056 | C22H29ClN2O | C10-2H |
| 375.2213 | C22H31ClN2O | C10 |

### Example 5: Antibody Method for Detecting RASP

### Summary:

Tear protein aldehyde level in the presence of absence of reproxalap was tested with both HABA/avidin/biotin method and streptavidin plate/lysozyme antibody method. In the HABA/avidin/biotin assay, tear protein is treated with Rx drug or placebo, conjugated with amine-PEG2-biotin and mixed with HABA/Avidin Premix. Unexpectedly, signal from the tear only control was as low as the PBS control. However, as expected, the placebo and drug groups were both higher than the PBS control. In the streptavidin plate/lysozyme antibody assay, tear protein was treated with reproxalap or placebo, conjugated with amine-PEG2-biotin, bound to a streptavidin-coated plate, and detected with lysozyme antibody-HRP. The tear only control was higher than the PBS control as expected, but the placebo and drug groups were both lower than the tear only control, indicating the interference of placebo/drug formulation component(s) to this assay. Due to these technical issues, no conclusion for the drug effect can be drawn. Further investigation is required to understand the possible interference of formulation components to aldehyde detection. However, the below procedures represent useful starting points for antibody-based assays for detecting RASP conjugates in a sample.

### HABA/Avidin/Biotin Assay Protocol:

Reagent List:
Drug Product
Placebo
PBS (Avoid buffers containing potassium (such as Modified Dulbecco's PBS), which will cause precipitation in the assay)
0.1M Acetic Acid, 5M NaCNBH₃
5 mM Amine-PEG2-Biotin Stock in H₂O
Biotinylated HRP (p.c.). Prepare the Biotinylated HRP at 1 mg/mL by adding the appropriate volume of ultrapure water (5 mL) to the vial (5 mg). Mix with a pipette tip and allow it to solubilize. Complete solubilization requires approximately 5 minutes at room temperature. Store solution in single-use volumes (i.e. 120 uL) at −20 C until ready to use.

Sample List:
3× tear+drug (100 uM biotin)
3× tear+placebo (100 uM biotin)
3× tear+PBS (100 uM biotin)
1×PBS Control (same process, with 100 uM biotin)

Day 1:

1. Preincubate 40 uL drug (Drug Product) or vehicle (Placebo) with 10 uL tear (containing ˜100 uM lysozyme plus other protein) for 2 hours at 30 C. Incubate with No HNE in both drug and placebo
2. Wash Away unreacted drug: dialyze in 1 L of PBS+0.1% Triton (7 KD filters) for 1 h. Note: Use separate reservoirs for placebo vs drug!
3. Wash Away unreacted drug and surfactant: dialyze in 1 L of Sigma PBS (7 KD filters) overnight.

Day 2:

4. Transfer retentate (˜100 uL) to new labeled tubes.
5. Bring retentate to 0.1M Acetic Acid (10 uL), incubate 60 minutes (cyclize the drug conjugates)
6. Spike the sample with 2 uL of sodium cyanoborohydride solution (5M in 0.1M acetic acid) (quench imines to amines), incubate 120 minutes.
7. Spike the samples with 2 uL of 5 mM Amine-PEG2-Biotin (100 uM biotin final).
8. Incubate 2 hours at 30 C.
9. Wash away unreacted biotin: dialyze in 1 L of PBS+0.1% Triton (7 KD filters) for 1 h.
10. Wash away unreacted biotin and surfactant: dialyze in 1 L of Sigma PBS (7 KD filters) at 4 C overnight.

Day 3

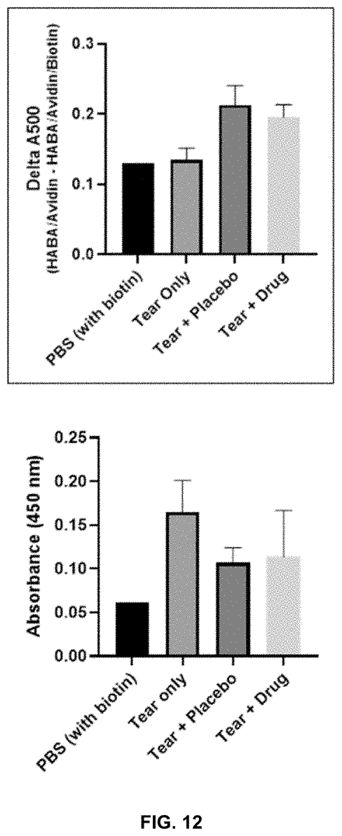

11. Transfer this biotinylated sample (retentate) to Step 15.
12. Equilibrate the HABA/Avidin Premix to room temperature.
13. Add 100 μL of ultrapure water to one microtube of the HABA/Avidin Premix. Mix with pipette tip.
14. Pipette 140 μL of PBS into a microplate well.
15. Add 20 μL of the HABA/Avidin Premix solution from step 12 to the PBS in the well. Place microplate on an orbital shaker or equivalent to mix.
16. Measure the absorbance of the solution in the well at 500 nm and record the value as A500 HABA/avidin. (high)
17. Add all ˜100 μL of biotinylated sample or 40 uL of Biotinylated HRP (positive control) to the well containing the HABA/avidin reaction mixture. Mix as described above.
18. Measure the absorbance of the solution in the well at 500 nm and record the value as A500 HABA/avidin/biotin sample once the value remains constant for at least 15 seconds. (low)
19. The relative level of aldehyde is determined by subtracting A500 HABA/avidin/biotin value from A500 HABA/avidin value.
Results: See FIG. 12 (top).

### Streptavidin Plate/Lysozyme Antibody Assay Protocol

Reagent List:
Drug Product
Placebo
PBS
5M NaCNBH₃, freshly dissolved in 0.1M Acetic Acid
5 mM Amine-PEG2-Biotin Stock in H₂O

Sample List:
3× tear+drug (no HNE)
3× tear+placebo (no HNE)
3× tear+PBS (same process)
1×PBS Control (same process)

Day 1:

1. Preincubate 40 uL drug (Drug Product) or vehicle (Placebo) with 10 uL tear (containing ˜100 uM lysozyme plus other protein) for 1.5 hour at 30 C. Incubate with No HNE in both drug and placebo.
2. Wash away unreacted drug: dialyze 1× in PBS with 0.2% Tween 80 for 3 h.
3. Wash away unreacted drug and surfactant: dialyze 1× in PBS overnight.

Day 2:

4. Bring retentate (˜100 uL) to 0.1M Acetic Acid, incubate 30 minutes (cyclize the drug conjugates) (+10 uL)
5. Spike the sample with 2 uL of sodium cyanoborohydride solution (freshly dissolved, 5M in 0.1M acetic acid).
6. Spike the samples with 1 uL of 5 mM Amine-PEG2-Biotin (50 uM biotin final).
7. Incubate 2 hours at 30 C.
8. Wash away unreacted biotin: dialyze 1× in PBS with 0.2% Tween 80 for 2-3 h.
9. Wash away unreacted biotin and surfactant: dialyze 1× with PBS overnight.

Day 3:

10. Transfer samples to a streptavidin-coated plate. Incubate at r.t. for 1-2 h.
11. Wash each well 3× with PBS (200 uL).
12. Add 100 ul of Anti-Lysozyme antibody-HRP (ab197705) (1:1000 in 1% BSA/PBS).
13. Incubate at room temperature for 2 h.
14. Wash each well 3× with PBS with 0.1% Triton.
15. Add 1004, of 1-Step Turbo TMB-ELISA to each well.
16. Incubate for 5-30 minutes at room temperature. (10 min)
17. Add 1004, of Stop Solution (1M H₂SO₄) to each well.
18. Transfer to a clear bottom plate.
19. Measure the absorbance at 450 nm.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

All publications, patents, patent applications and other documents cited in this application are hereby incorporated by reference in their entireties for all purposes to the same extent as if each individual publication, patent, patent application or other document were individually indicated to be incorporated by reference for all purposes.

We claim:

1. A method of assessing effectiveness of an aldehyde trapping agent in treating dry eye disease in a subject, comprising:
administering an aldehyde trapping agent to a subject with dry eye disease;
measuring the level of an aldehyde marker of oxidative stress present in the eye of the subject; and
comparing the measured level of the aldehyde marker of oxidative stress to level of aldehyde marker of oxidative stress in an appropriate control;
wherein a reduction in level of aldehyde marker of oxidative stress indicates effectiveness of the aldehyde trapping agent in treating dry eye disease.

2. The method of claim 1, wherein the aldehyde marker of oxidative stress in dry eye disease is formaldehyde, acetaldehyde, acrolein, glyoxal, methylglyoxal, hexadecanal, octadecanal, hexadecenal, succinic semi-aldehyde, malondialdehyde, 4-hydroxynonenal (4-HNE or HNE), 4-hydroxy-2E-hexenal, 4-hydroxy-2E,6Z-dodecadienal, retinaldehyde, leukotriene B4 aldehyde, or octadecenal.

3. The method of claim 2, wherein the aldehyde marker of oxidative stress in dry eye disease is malondialdehyde or 4-hydroxynonenal.

4. The method of claim 3, wherein the aldehyde marker of oxidative stress in dry eye disease is malondialdehyde.

5. The method of any one of claims 1 to 4, wherein the measuring of the level of an aldehyde marker of oxidative stress is conducted on a sample of tears obtained from the subject.

6. The method of any one of claims 1 to 5, wherein the appropriate control is level of the aldehyde marker of oxidative stress in the eye of the subject prior to administration of the aldehyde trapping agent or level of the aldehyde marker of oxidative stress in subjects diagnosed with dry eye disease.

7. The method of any one of claims 1 to 6, wherein the level of the aldehyde marker of oxidative stress measured is in the form of adducts of the aldehyde marker of oxidative stress present in the eye.

8. The method of claim 7, wherein the adducts comprise stable adducts formed with proteins in the eye.

9. The method of claim 8, wherein the adducts comprise adducts formed with malondialdehyde.

10. The method of claim 8, wherein the adducts comprise adducts formed with 4-hydroxynonenal.

11. The method of any one of claims 7 to 10, wherein the adducts are detected by enzyme linked immunosorbent assay (ELISA).

12. The method of any one of claims 1 to 11, wherein at least about 15% or greater reduction in level of the aldehyde marker of oxidative stress compared to control level is indicative of effectiveness of the aldehyde trapping agent in treating dry eye disease.

13. The method of claim 12, wherein at least about 20% or greater reduction in level of the aldehyde marker of oxidative stress compared to control level is indicative of effectiveness of the aldehyde trapping agent in treating dry eye disease.

14. The method of claim 12, wherein at least about 25% or greater reduction in level of level of the aldehyde marker of oxidative stress compared to control levels is indicative of effectiveness of the aldehyde trapping agent in treating dry eye disease.

15. The method of any one of claims 7 to 14, wherein the control level is a mean malondialdehyde adduct concentration of about 14,000 pmol/L to about 14,900 pmol/L as measured according to Example 2.

16. The method of claim 15, wherein a measured level of malondialdehyde adduct concentration of about 12,000 pmol/L or lower as measured according to Example 2 in human tears is indicative of effectiveness of the aldehyde trapping agent in treating dry eye disease.

17. The method of any one of claims 1 to 16, wherein the aldehyde trapping agent is reproxalap.

18. The method of claim 17, wherein the reproxalap is administered topically to the eye at a concentration of 0.1% to 0.5% w/v.

19. The method of claim 18, wherein the reproxalap is administered topically to the eye at a concentration of 0.25% w/v.

20. The method of claim 18, wherein the reproxalap is administered topically to the eye at a concentration of 0.1% w/v.

21. The method of claim 18, wherein the reproxalap is administered topically to the eye at a concentration of 0.5% w/v.

22. The method of any one of claims 17 to 21, wherein the reproxalap is administered as an admixture with a pharmaceutically acceptable excipient, wherein the excipient is a cyclodextrin selected from sulfobutylether-β-cyclodextrin and hydroxypropyl-β-cyclodextrin.

23. The method of claim 22, wherein the pharmaceutically acceptable excipient is sulfobutylether-β-cyclodextrin.

24. The method of any one of claims 22 to 23, wherein the cyclodextrin is present at 5% to 20% w/v.

25. The method of claim 24, wherein the cyclodextrin is present at 7% to 11% w/v.

26. The method of claim 25, wherein the reproxalap is at a concentration of 0.25% w/v and the cyclodextrin is present at 7% w/v.

27. The method of claim 25, wherein the reproxalap is at a concentration of 0.25% w/v and the cyclodextrin is present at 11% w/v.

28. The method of any one of claims 17 to 27, wherein the level of the aldehyde marker of oxidative stress is measured after 28 days of treatment.

29. The method of any one of claims 17 to 28, wherein the treatment comprises topically administering the aldehyde trapping agent up to six times a day.

30. The method of claim 29, wherein the treatment comprises topically administering the aldehyde trapping agent four times a day (QID).

31. The method of any one of claims 17 to 27, wherein the treatment comprises an initiation phase and/or exacerbation phase, followed by a maintenance phase.

32. The method of claim 31, wherein the treatment in the initiation phase and/or exacerbation phase comprises topically administering the aldehyde trapping agent to the eye four times a day (QID), and the treatment in the maintenance phase comprises topically administering the aldehyde trapping agent to the eye four times a day (QID) or two times a day (BID).

33. The method of any one of claims 21 to 32, wherein the measuring the levels of the aldehyde marker of oxidative stress is during or following the initiation and/or exacerbation phase.

34. The method of any one of claims 21 to 32, wherein the measuring the levels of the aldehyde marker of oxidative stress is in the maintenance phase.

35. A method of treating dry eye disease in a subject comprising:
(i) measuring the level of an aldehyde marker of oxidative stress in the eye of a subject with dry eye disease prior to treatment;
(ii) treating the subject with an aldehyde trapping agent, wherein the aldehyde trapping agent is reproxalap and wherein the reproxalap is administered topically to the eye; and
(iii) measuring the level of the aldehyde marker of oxidative stress in the eye of the subject following treatment;
wherein the subject is treated with a lower dosing frequency of reproxalap for a reduction of greater than about 20% in the measured level of the aldehyde marker of oxidative stress, and
wherein the subject is treated with the same or higher dosing frequency of reproxalap for a reduction of about 20% or less in the measured level of the aldehyde marker of oxidative stress.

36. The method of claim 35, wherein the aldehyde marker of oxidative stress in dry eye disease is formaldehyde, acetaldehyde, acrolein, glyoxal, methylglyoxal, hexadecanal, octadecanal, hexadecenal, succinic semi-aldehyde, malondialdehyde, 4-hydroxynonenal (4-HNE or HNE), 4-hydroxy-2E-hexenal, 4-hydroxy-2E,6Z-dodecadienal, retinaldehyde, leukotriene B4 aldehyde, or octadecenal.

37. The method of claim 35, wherein the aldehyde marker of oxidative stress in dry eye disease is malondialdehyde or 4-hydroxynonenal.

38. The method of claim 37, wherein the aldehyde marker of oxidative stress in dry eye disease is malondialdehyde.

39. The method of any one of claims 35 to 38, wherein the measuring of the level of an aldehyde marker of oxidative stress is conducted on a sample of tears obtained from the subject.

40. The method of any one of claims 35 to 39, wherein the level of the aldehyde marker of oxidative stress measured is in the form of adducts of the aldehyde marker of oxidative stress present in the eye.

41. The method of claim 40, wherein the adducts comprise stable adducts formed with proteins in the eye.

42. The method of claim 40 or 41, wherein the adducts comprise adducts formed with malondialdehyde.

43. The method of claim 40 or 41, wherein the adducts comprise adducts formed with 4-hydroxynonenal.

44. The method of any one of claims 40 to 43, wherein the adducts are detected by enzyme linked immunosorbent assay (ELISA).

45. The method of any one of claims 35 to 44, wherein the reproxalap is administered topically at a concentration of 0.1% to 0.5%% w/v.

46. The method of claim 45, wherein the reproxalap is administered topically to the eye at a concentration of 0.25% w/v.

47. The method of claim 45, wherein the reproxalap is administered topically to the eye at a concentration of 0.1% w/v.

48. The method of claim 45, wherein the reproxalap is administered topically to the eye at a concentration of 0.5% w/v.

49. The method of any one of claims 35 to 48, wherein the reproxalap is administered as an admixture with pharmaceutically acceptable excipient, wherein the excipient is a cyclodextrin selected from sulfobutylether-β-cyclodextrin and hydroxypropyl-β-cyclodextrin.

50. The method of claim 49, wherein the pharmaceutically acceptable excipient is sulfobutylether-β-cyclodextrin.

51. The method of any one of claims 49 to 50, wherein the cyclodextrin is present at about 5% to 20% w/v.

52. The method of claim 51, wherein the cyclodextrin is present at 7% to 11% w/v.

53. The method of claim 52, wherein the reproxalap is at a concentration of 0.25% w/v and the cyclodextrin is present at 7% w/v.

54. The method of claim 52, wherein the reproxalap is at a concentration of 0.25% w/v and the cyclodextrin is present at 11% w/v.

55. The method of any one of claims 35 to 54, wherein the reproxalap is administered up to six times a day.

56. The method of claim 55, wherein the reproxalap is administered four times a day (QID).

57. The method of any one of claims 35 to 56, wherein the subject is treated with a lower dosing frequency of reproxalap for a reduction of about 25% or greater in the measured level of the aldehyde marker of oxidative stress.

58. The method of any one of claims 35 to 57, wherein the treatment comprises an initiation and/or exacerbation phase, and a maintenance phase.

59. The method of claim 58, wherein the measuring following treatment is done during or following the initiation and/or exacerbation phase.

60. The method of any one of claims 58 to 59, wherein reproxalap is administered four times a day (QID) in the initiation and/or exacerbation phase.

61. The method of any one of claims 58 to 60, wherein the subject is treated with a lower dosing frequency of reproxalap in the maintenance phase for a reduction of greater than about 20% in the measured level of the aldehyde marker of oxidative stress in the initiation and/or exacerbation phase.

62. The method of claim 61, wherein the subject is treated with a lower dosing frequency of reproxalap in the maintenance phase for a reduction of about 25% or greater in the measured level of the aldehyde marker of oxidative stress in the initiation and/or exacerbation phase.

63. The method of any one of claims 61 to 62, wherein the lower dosing frequency is two times a day (BID).

64. A method of selecting a subject for treatment of dry eye disease, comprising: measuring the level of an aldehyde marker of oxidative stress in an eye of a subject suspected of having dry eye disease, wherein a measured level of at least about 2 fold or greater level of the aldehyde marker of oxidative stress as compared to level of aldehyde marker of oxidative stress in subjects without dry eye disease is indicated for treatment.

65. The method of claim 64, wherein the aldehyde marker of oxidative stress in dry eye disease is formaldehyde, acetaldehyde, acrolein, glyoxal, methylglyoxal, hexadecanal, octadecanal, hexadecenal, succinic semi-aldehyde, malondialdehyde, 4-hydroxynonenal (4-HNE or HNE), 4-hydroxy-2E-hexenal, 4-hydroxy-2E,6Z-dodecadienal, retinaldehyde, leukotriene B4 aldehyde, or octadecenal.

66. The method of claim 65, wherein the aldehyde marker of oxidative stress is malondialdehyde or 4-hydroxynonenal.

67. The method of any one of claims 64 to 66, wherein the level of the aldehyde marker of oxidative stress measured is in the form of adducts of the aldehyde marker of oxidative stress present in the eye.

68. The method of claim 67, wherein the adducts comprise stable adducts formed with proteins in the eye.

69. The method of claims 67 to 68, wherein the adducts comprise adducts formed with malondialdehyde.

70. The method of claims 67 to 68, wherein the adducts comprise adducts formed with 4-hydroxynonenal.

71. The method of any one of claims 67 to 70, wherein the measuring is of adducts present in the tears of the subject.

72. The method of any one of claims 67 to 70, wherein the adducts are detected by enzyme linked immunosorbent assay (ELISA).

73. The method of any one of claims 64 to 72, wherein the treatment is with an aldehyde trapping agent.

74. The method of claim 73, wherein the aldehyde trapping agent is reproxalap.

75. The method of claim 74, wherein the reproxalap is administered topically to the eye at a concentration of 0.1% to 0.5% w/v.

76. The method of claim 75, wherein the reproxalap is administered topically to the eye at a concentration of 0.25% w/v.

77. The method of claim 75, wherein the reproxalap is administered topically to the eye at a concentration of 0.1% w/v.

78. The method of claim 75, wherein the reproxalap is administered topically to the eye at a concentration of 0.5% w/v.

79. The method of any one of claims 74 to 78, wherein the reproxalap is administered as an admixture with pharmaceutically acceptable excipient, wherein the excipient is a cyclodextrin selected from sulfobutylether-β-cyclodextrin and hydroxypropyl-β-cyclodextrin, or a pharmaceutically acceptable salt thereof.

80. The method of claim 79, wherein the pharmaceutically acceptable excipient is sulfobutylether-β-cyclodextrin or a pharmaceutically acceptable salt thereof.

81. The method of any one of claims 79 to 80, wherein the cyclodextrin is present at 5% to 20% w/v.

82. The method of claim 81, wherein the cyclodextrin is present at 7% to 11% w/v.

83. The method of claim 82, wherein the reproxalap is at a concentration of 0.25% w/v and the cyclodextrin is present at 7% w/v.

84. The method of claim 82, wherein the reproxalap is at a concentration of 0.25% w/v and the cyclodextrin is present at 11% w/v.

85. The method of any one of claims 64 to 84, wherein the level of aldehyde marker of oxidative stress is in the form of malondialdehyde adduct and wherein a measured level malondialdehyde adduct of at least about 3.4 fold or greater as compared to level of malondialdehyde adduct in subjects without dry eye disease is indicated for treatment.

86. The method of any one of claims 64 to 85, further comprising the step of treating the dry eye disease in the subject if the level of the aldehyde marker of oxidative stress is indicated for treatment.

87. The method of any one of claims 64 to 86, wherein the level of the aldehyde marker of oxidative stress is indicated for treatment if it is at least about 3-fold higher than the level of aldehyde marker of oxidative stress in subjects without dry eye disease.

88. The method of any one of claims 7 to 10, 40 to 43, or 67 to 70, wherein the adducts are detected by LC-MS, ultraviolet spectrometry (UV), HPLC, mass spectrometry (MS), monoclonal antibody detection assay, gas chromatography (GC), GC/MS, GC/flame ionization detector (FID), capillary electrophoresis with amperometric detection (CE-AD), liquid chromatography/fluorescence detection, or a combination thereof.
